(19) United States
(12) Patent Application Publication
(10) Pub. No.: US 2018/0362449 A1
(43) Pub. Date: Dec. 20, 2018

(54) THE EXOCYST AS A NOVEL DRUG TARGET OF ENDOSIDIN2 AND APPLICATION AS A THERAPEUTIC

(71) Applicant: The Regents of the University of California, Oakland, CA (US)

(72) Inventors: Natasha V. Raikhel, Pasadena, CA (US); Glenn R. Hicks, Riverside, CA (US); Chunhua Zhang, West Lafayette, IN (US)

(73) Assignee: The Regents of the University of California, Oakland, CA (US)

(21) Appl. No.: 15/756,804

(22) PCT Filed: Sep. 15, 2016
(86) PCT No.: PCT/US16/52020

§ 371 Date: Mar. 1, 2018

Related U.S. Application Data
(60) Provisional application No. 62/219,029, filed on Sep. 15, 2015.

(51) Int. Cl. C07C 243/38; G01N 33/68; A61K 31/166; C07D 495/04

(52) U.S. Cl. CPC C07C 243/38; G01N 33/68; G01N 2500/20; C07D 495/04; G01N 2500/04; A61K 31/166

(57) ABSTRACT

A method of altering exocytosis in a plant or animal cell is provided. The method includes exposing the cell to a compound that binds to an EXO70 protein isoform. Also provided is a method of treating diabetes or cancer in a subject in need thereof which includes administering to the subject an effective amount of a compound that binds to an EXO70 protein isoform. In addition, a method of screening for a substance that alters exocytosis in a plant or animal cell is provided, and analogs of compound Endosidin2 are also provided.

### CROSS-REFERENCE TO RELATED APPLICATIONS

This application claims the benefit of U.S. Provisional Patent Application No. 62/219,029, filed on Sep. 15, 2015, which is incorporated by reference herein.

### STATEMENT REGARDING FEDERALLY SPONSORED RESEARCH OR DEVELOPMENT

This invention was made with Government support under Grant DE-FG02-02ER15295 from the United States Department of Energy. The Government has certain rights in this invention.

### BACKGROUND

### Field of the Invention

The invention relates to the alteration of exocytosis and endocytic recycling.

### Related Art

The exocyst complex regulates the last steps of exocytosis, which is essential to organisms across kingdoms. In humans, its dysfunction is correlated with several significant diseases such as diabetes and cancer progression. Investigation of the dynamic regulation of the evolutionarily conserved exocyst-related processes using mutants in genetically tractable organisms such as Arabidopsis thaliana is limited by the lethality or the severity of phenotypes.

The exocyst is a protein complex involved in multiple processes in plants and animals including exocytosis and autophagy among others. It is composed of eight subunits (Exo70, Sec3, Sec8, Sec6, Exo84, Sec10, Sec15). In addition, there are many known interacting proteins with the complex including GTPases¹².

The EXO70 protein is a component of the evolutionary conserved octameric exocyst complex that tethers post-Golgi vesicles to the plasma membrane prior to SNARE-mediated membrane fusion¹. As an important component of the exocyst complex that mediates exocytosis, EXO70 regulates, for example, neurite outgrowth, epithelial cell polarity establishment, cell motility, and cell morphogenesis in animal cells²⁻⁶. In plants, EXO70 proteins participate in polarized pollen tube growth, root hair growth, deposition of cell wall material, cell plate initiation and maturation, defense and autophagy⁷⁻¹². In humans, EXO70 mediates the trafficking of the glucose transporter Glut4 to the plasma membrane which is stimulated by insulin and involved in the development of diabetes¹³. A specific isoform of human EXO70 is also involved in cancer cell invasion¹³⁻¹⁵.

### SUMMARY

The small molecule Endosidin2 (ES2) has been discovered to bind to the EXO70 subunit of the exocyst complex, resulting in inhibition of exocytosis and endosomal recycling in both plant and human cells and enhancement of plant vacuolar trafficking. An EXO70 protein with a C-terminal truncation results in dominant ES2 resistance, uncovering possible distinct regulatory roles for the N-terminus of the protein. This study provides not only a valuable tool in studying exocytosis regulation but also offers a new target for drugs aimed at addressing human disease.

Endosidin2 (ES2) was identified from a plant-based chemical screen. It is demonstrate that its target is the EXO70 subunit of the exocyst and ES2 is active in plants and mammalian systems. Significantly, no inhibitor of the exocyst complex has been reported, yet such compounds could be important for understanding the basic mechanisms of exocyst-mediated processes, for modifying secretion in biotechnological applications, and as new drugs to control exocyst-related diseases.

In one aspect, a method of altering exocytosis and/or or endocytic recycling in a plant, fungal, or animal cell is provided. The method includes exposing the cell to a compound that modifies the activity of the exocyst complex. In some embodiments, the method includes exposing the cell to a compound that modifies exocytosis and/or endocytic recycling by binding to an exocyst complex or an exocyst complex subunit of the cell.

In another aspect, a method of screening for a substance or a compound for altering exocytosis and/or endocytic recycling in a plant, fungal or animal cell is provided. The method includes identifying or detecting a substance or a compound that binds to a plant, fungal or animal exocyst complex. In some embodiments, the method includes detecting or measuring the binding of a test compound to an exocyst complex or to a subunit of an exocyst complex in a cell-free system, or identifying a test compound that binds to an exocyst complex or to an exocyst complex subunit in a cell-free system. After the detecting, measuring or identifying, the method can further include assaying the test compound for its effect on exocytosis and endocytic recycling.

In a further aspect, a method of treating diabetes or cancer in a subject in need thereof is provided. The method includes administering to the subject an effective amount of a compound that modifies the activity of the exocyst complex. In some embodiments, the method includes administering to the subject an effective amount of a compound that alters exocytosis and/or endocytic recycling by binding to an exocyst complex or to a subunit of an exocyst complex.

In any embodiment of the method of altering exocytosis and/or or endocytic recycling, method of screening for a substance or compound for altering exocytosis and/or endocytic recycling, and method of treating diabetes or cancer: a) the subunit can be an EXO70 protein isoform; b) the EXO70 protein isoform can be EXO70A1; c) the compound can promote or inhibit exocyst complex activity; d) the compound can bind to the C-terminal portion of EXO70A1; e) the compound can bind to a cavity in the C-terminal portion of EXO70A1; f) the compound can be Endosidin2 (ES2) or an analog thereof; or g) any combination of a)-f).

In embodiments that include an analog of ES2, the analog can be based on N′-benzylidenebenzohydrazide with substitutions and/or other modifications on one or both phenyl rings.

In any embodiment, the analog can be: A) an N′-benzylidenebenzohydrazide analog of compound ES2, wherein the analog includes (i) a substituted or non-substituted iodine-containing phenyl group of ES2, or (ii) a substituted or non-substituted fluorine-containing benzoic ring of ES2, or (iii) both (i) and (ii); B) the benzoic ring can lack the fluorine present in ES2; C) the analog can bind to an exocyst complex or to a subunit of an exocyst complex; D) the analog can bind to EXO70A1; E) the analog can bind to the C-terminal portion of EXO70A1; F) the analog can bind to a cavity in the C-terminal portion of EXO70A1; G) the analog (i) can promote or inhibit exocyst complex activity, (ii) can alter exocytosis and/or endocytic recycling in a plant or animal cell, (iii) can inhibit exocytosis and/or endocytic recycling in the plant or animal cell, or (iv) any combination of (i)+(ii), (i)+(iii), (ii)+(iii), or (i)+(ii)+(iii); H) or any combination of A)-G).

Also provided in another aspect is any compound, such as an ES2 analog, that modifies or alters exocyst complex activity, or that binds to a plant, fungal or animal exocyst complex or a subunit of an exocyst complex. The compound can be a compound as described above for the method of altering exocytosis and/or or endocytic recycling, method of screening for a substance or compound for altering exocytosis and/or endocytic recycling, and method of treating diabetes or cancer.

### BRIEF DESCRIPTION OF THE DRAWINGS

For a more complete understanding of the present invention, reference is now made to the following descriptions taken in conjunction with the accompanying drawings, in which:

FIG. 1 is a panel showing that ES2 inhibits trafficking to the plasma membrane and enhances trafficking to the vacuole in Arabidopsis.
A. ES2 molecular structure.
B. Time course images of PIN2 localization in root epidermal cells treated with 0.5% DMSO at time 0 (top image) and time 120 minutes (bottom image) under normal light conditions. The lines in the cross section of the images show the location of plot profile shown in b.
C. Plot profile of the lines shown in images in panel a. The fluorescence intensity along the line at time 0 is shown in green and along the line at time 120 minutes shown in red. Panels a and b show that the fluorescence intensity of PIN2 at the plasma membrane is not significantly altered over a time course of 2 hours.
D. Time course images of PIN2 localization in root epidermal cells treated with 40 μM ES2 at time 0 (top image) and time 120 minutes (bottom image) under normal light conditions. The lines in the cross section of the images show the location of the plot profile shown in d.
E. Plot profile of the lines shown in images in panel d. The fluorescence intensity along the line at time 0 is shown in green and along the line at time 120 minutes shown in red. Panels c and d show that the fluorescence intensity of PIN2 at the plasma membrane is reduced after 2 hours of ES2 treatment.
F. ES2 enhances trafficking to the vacuole. Images show root cells from PIN2::PIN2:GFP seedlings treated with 0.5% DMSO (left) or 40 μM ES2 (right) for 2 hours in the dark.
G. Box plot showing the size distribution of PIN2 agglomerations after ES2 treatment.
H. ES2-induced PIN2 agglomerations colocalize with ARA7/RabF2b. Top panels, images from PIN2::PIN2:GFP;ARA7/RabF2b:mRFP root cells treated with 0.5% DMSO, showing GFP channel, mRFP channel and merged channel from left to right. Bottom panels, images from PIN2::PIN2:GFP;ARA7/RabF2b:mRFP root cells treated with 40 μM ES2, showing GFP channel, mRFP channel and merged channel from left to right.
I. ES2 reduces recycling efficiency of BFA-induced PIN2 positive membrane aggregates. Left panel image shows PIN2 compartments induced by BFA treatment. Middle panel image shows PIN2 localization after 90 minutes of recovery in normal 0.5×MS media. Right panel image shows PIN2 localization after 90 minutes of recovery in 0.5×MS media containing 40 μM ES2. Arrows show the residual PIN2 aggregates.
1J. Quantification of cells containing BFA compartments after recovery in media containing DMSO or ES2. All bars=10 μm

FIG. 2 is a panel showing that ES2 interacts with EXO70A1 subunit of the exocyst complex.
A to E. Structure and bioactivity of ES2 and its synthesized analogs. Left panels are the structures and right panels are the PIN2 localization after treatment with 40 μM analogs. Bio-688 (d, active) and Bio-680 (e, inactive) were used in pull down assays.
F. Western blot detection of EXO70A1 in the resin from the pull down assays using anti-EXO70A1 antibody. Lane 1, 1% of the input sample. Lane 2, resin from Bio-688 sample. Lane 3, resin from Bio-680 sample. Lane 4, resin from free Biotin sample.
G. Quantification of signal intensity in lane 2, lane 3 and lane 4 in panel f.
2H. DARTS assay shows that ES2 protects EXO70A1, but not actin, from degradation. Top panels, western blot of DARTS samples treated with different concentrations of pronase using anti-EXO70A1 and anti-actin antibodies. The chart at the bottom shows the signal intensity ratio between ES2 and DMSO using antibodies against EXO70A1 and actin under different dilutions of pronase. At 10,000 and 3,000 dilutions, EXO70A1 protein was obviously protected from degradation by ES2. The error bars represent standard errors of two independent experiments.
I. Coomassie staining of purified EXO70A1 protein (aa75 to end) used for STD-NMR experiments.
J. ES2 ¹H assignment and the spectrum of STD-NMR using purified EXO70A1 protein and ES2. Red, green and blue colors represent off resonance, on resonance and different spectra respectively. The arrow indicates a spectral from the buffer component present in the sample. The signal from the buffer is not present in the STD-NMR spectrum.
Bars=10 μm
K. STD-NMR binding curves of EXO70A1 titrated with different concentrations of ES2 (red) and analog8 (blue). Red circles represent STD amplification factors of ES2H1 through H7 dosed with ES2. Blue squares represent STD amplification factors of analog8H2a, H2b and CH3 dosed with analog8. Solid lines represent the non-linear fit curves and the dotted lines represent 95% confidence interval of the fit.
L. Thermophoresis binding curves of NT-647 labeled EXO70A1 titrated with different concentrations of ES2 (red) and analog8 (blue). Red circles and blue squares represent normalized fluorescence of EXO70A1 dosed with ES2 and analog8, respectively, from each replicate experiment. Solid lines represent the non-linear fit curves and the dotted lines around the fit represent 95% confidence interval of the fit.

FIG. 3 is a panel showing that EXO70A1 N-terminal peptide contributes to ES2 resistance.
A. Heterozygous mutant allele of exo70a1-3 displays resistance to ES2.
B. Wild-type seedlings from EXO70A1 heterozygous progeny expressing PIN2::PIN2:GFP treated with 40 uM ES2 for two hours.
C. Heterozyous seedlings from EXO70A1 heterozygous progeny expressing PIN2::PIN2:GFP treated with 40 uM ES2 for two hours.
D. Heterozygous mutant allele of exo70a1-3 (N=370 cells from 16 seedlings) has smaller PIN2 agglomerations compare with wildtype seedlings (N=241 cells from 12 seedlings) in the same segregating population (p<0.001).
E. Heterozygous mutant allele of exo70a1-3 (N=370 cells from 16 seedlings) has less numbers of PIN2 agglomerations compare with wildtype seedlings (N=241 cells from 12 seedlings) in the same population (p<0.001).
3F. Diagram showing EXO70A1 gene organization and the location of T-DNA insertion in exo70a1-3 allele. The arrows mark locations and orientations of the primers used for allele characterization in panel e.
G. RT-PCR shows that T-DNA insertion causes accumulation of truncated EXO70A1 mRNA in homozygous exo70a1-3 seedlings. EXO70A1 mRNA sequence 3′ of T-DNA insertion site was not detected.
H. MS spectra of extracted ion chromatograms of m/z 756.43,3+ for the peptide spanning amino acid 90 to 109 of EXO70A1 from protein gel bands of wild-type or exo70a1-3. EXO70A1 N-terminal peptide (amino acids 90 to 109) was detected in the 70 kd region but not the 25 kd region from wildtype plants; the same peptide was detected in the 25 kd region but not the 70 kd region from exo70a1-3 homozygous plants.
I. Transgenic plants that express EXO70A1 N-terminal peptide (amino acids 1 to 231) are less sensitive to ES2 in root elongation. The transgenic plants have longer roots in comparison with PIN2::PIN2:GFP line on 20 μM ES2 plates.
J. The ratio of root growth on 30 μM ES2 relative to 0.5% DMSO in different genotypes at different time point after being transferred. Col, exo70a1-3 heterozygous and EXO70A1N seeds were grown on ½ MS agar plates for 3 days and the seedlings were then transferred to ½ MS agar plates containing 0.5% DMSO or 30 μM ES2. The absolute root growth of each seedling after transfer was calculated at different time point. The ratio of root growth on 30 μM ES2 relative to the root growth on DMSO was calculated for each genotype at each time point. * represents significant difference to Col in T-TEST (P<0.05).
Bars in b and c=10 μm.
K. An expanded view of the MS spectra in FIG. 3H, showing details of the 25kd_Wild-type, 70kd_Wild-type, and 70kd_exo70A1-3 spectra.

FIG. 4 is a panel showing that ES2 inhibits EXO70A1 dynamics in Arabidopsis root cells.
A. EXO70A1 has a polar localization pattern on the outer lateral side of the root epidermal cells in the transition zone, and this pattern is altered by ES2 treatment. Localization of EXO70A1 in the transition zone after 2 hours or 4 hours of treatment with DMSO (top panels) or ES2 (bottom panels) was shown in right panels.
B. Plot profiles of fluorescence intensity across the yellow lines in DMSO treated sample as shown in panel a.
C. Plot profile of fluorescence intensity across the red line in ES2 treated sample as shown in panel a.
D. Images of fluorescence recovery after photobleaching (FRAP) analysis in root hair cells treated with 0.05% DMSO (top) or 4 μM ES2 (bottom). Pre-bleach represents the fluorescence before photo bleaching. Pb represents the time point of post bleach recovery. The yellow shapes in Pre-bleach images indicate the regions that were manually selected for photo bleaching.
E. Plot of relative fluorescence intensity to pre-bleaching at different time points in ES2 treated and control cells shown in panel d.
Bars=10 μm.

FIG. 5 is a panel showing that ES2 targets EXO70 to inhibit exocytosis in mammalian cells.
A. ES2 treatment inhibits the recycling of transferrin in Hela cells. Time course images of Hela cells transformed with Alexa488-transferrin that were chased in the presence of DMSO (top panels) or ES2 (bottom) panels.
B. The amount of transferrin retained in cells in panel a was measured using Image J and plotted in the bar charts. 100 cells were analyzed for each time. * p=0.038, **p<0.01.
C. ES2 inhibits plasma membrane localization of GFP-rEXO70 in Hela cells. Upon ES2 treatment, GFP-EXO70 is accumulated in intracellular compartments (right panel).
D. Coomassie Blue staining of purified rat EXO70 protein used for STD-NMR experiment.
E. The spectrum of STD-NMR experiment shows interaction between ES2 and rEXO70A1. The off resonance spectrum, on resonance spectrum and the different spectrum are shown in red, green and blue, respectively. The arrow indicates a spectral from the buffer component that does not show in the STD-NMR.
Bars=10 μm

FIG. 6 is a panel showing that EXO70A1 amino acids L596 and I613 ES2 participate in interaction with ES2.
A. Structure of EXO70A1. The structure is divided into three domains: N-terminal domain (blue), C-terminal domain (green) and Middle domain (cyan).
B. Structural superposition of EXO70A1 with mEXO70.
C. Conformation of EXO70A1-ES2 complex from docking simulation. ES2 (magenta) fits in the cavity that is formed by several amino acids (cyan). The two yellow circles highlight the two amino acids L596 and I613 that were mutated to A as shown in panel d.
D. Conformation of EXO70A1-L596A;I613A-ES2 complex from docking simulation. With the two mutations at L596 and I613, ES2 conformation is changed in compare with wild-type EXO70A1 protein.
E. STD-NMR profiles of EXO70A1 (bottom) and EXO70A1-L596A;I613A (top) with ES2 when using the same concentrations of protein and ES2. The spectrums are presented in the same scale. The integrity of individual STD-NMR spectrum is less in reactions with mutant protein in compare with the reactions using wild-type protein.
G. The ratio of STD-NMR spectrum integrity of different protons in ES2 between EXO70A1-L596A;I613A and EXO70A1 shown in c. In EXO70A1-L596A;I613A mutant protein, the integrities of STD-NMR spectrum in ES2 is reduced to about 20% of the wild-type EXO70A1 protein.
G. Thermophoresis binding curves of NT-647 labeled purified EXO70A1 and EXO70A1-L596A;I613A titrated with different concentrations of ES2. Red circles and blue squares represent the normalized fluorescence of EXO70A1 and EXO70A1-L596A;I613A, respectively, in each triplicate experiment. Solid lines represent the non-linear fit curves and the dotted lines around the fit curve represent 95% confidence interval of the fit.

FIG. 7 is a panel showing that ES2 inhibits polarized growth and gravitropic response.
A. Images of pollens that were germinated on media in the absence (left) or presence (right) of ES2. ES2 inhibits pollen tube growth and 16 μM ES2 inhibits germination. Bars=100 μm.
B. The length of pollen tubes that were germinated on media contains different concentrations of ES2. ES2 inhibits pollen tube elongation in a dosage-dependent manner.
C. Arabidopsis wildtype (Col) and PIN2::PIN2:GFP seedlings grown on DMSO or ES2 containing media for 7 days, showing reduced root elongation and agravitropic response on 40 μM ES2 media. Bars=1 cm.
D. The root gravity response angle and root length of Arabidopsis seedlings grown on different concentrations of ES2. Both root length and gravity response angle are affected by ES2 in a dosage dependent manner.
E. Root length of Arabidopsis wildtype seedlings grown on media containing 0.5% DMSO or 40 μM ES2 over time course.
F. Gravitropic response curve of seedlings grown on DMSO or 15 μM ES2, showing ES2 inhibition of seedling response to gravity stimulation.
G. ES2 inhibits root hair elongation. 1 and 2 show 5 days old seedlings grown on 0.5×MS media with 0.5% DMSO (1) and 40 μM ES2 (2). 3 and 4 show root hairs of seedlings grown on 0.5×MS 0.5% DMSO (3) or 40 μM ES2 (4). Bars in 1 and 2=1 mm. Bars in 3 and 4=0.2 mm.

FIG. 8 is a panel showing that ES2 affects cellular localization of PIN2, PIN1 (PIN1 with a GFP tag driven by PIN2 promoter) and BRI1 but does not affect SYP61 or ROP6, indicating that ES2 does not target the same pathway as ES1 or ES3. Bars=10 μm

FIG. 9 is a panel showing that ES2 is specific for certain plasma membrane proteins and does not disrupt cellular localization of known marker proteins for ER, Golgi or PVC. Bars=10 μm

FIG. 10 is a panel showing that ES2 molecule is stable under aqueous solution.
A. Predicted ES2 hydrolysis reaction.
B. H¹ NMR spectrum of ES2 in water over a time course of 7 days.
C to F. PIN2 localization in PIN2::PIN2:GFP seedlings treated with 0.5% DMSO (C), 40 μM ES2 (E), 40 μM 3-fluorobenzohydrazide (E) and 40 μM or 4-hydroxy-3-iodo-5-methoxybenzaldehyde (F) for two hours.

FIG. 11 is a panel showing Structure Activity Relationship analysis of ES2 molecule.
A. Structures of active ES2 analogs that induce PIN2 agglomerations after 2 hours of treatment at 40 μM.
B. Structures of inactive ES2 analogs that do not induce PIN2 agglomerations at 40 μM.

FIG. 12 is a panel of the ¹H NMR spectral assignment of ES2 and analog8 and buildup curve of STD-NMR.
A. gCOSY spectrum of ES2 in DMSO-d₆.
B. gNOESY spectrum of ES2 in DMSO-d₆
C. The STD amplification factor as a function of saturation time in ES2 and EXO70A1 binding reaction.
D. gCOSY spectrum of analog8 in DMSO-d₆.

FIG. 13 is a panel showing growth phenotypes of exo70a1-3 heterozygous and homozygous plants.
A. exo70a1-3 heterozygous plants have retarded growth, reduced silique size and reduced seeds yield. The pictures show wildtype and exo70a1-3 heterozygous mutant plant that are at the same age.
B. Homozygous exo70a1-3 seedlings have severe growth defects and are seedling lethal.

FIG. 14 is the MS/MS spectrum of m/z 756.43, 3+ for the peptide spanning amino acid 90 to 109 of EXO70A1.

FIG. 15 is a panel showing transgenic plants expressing EXO70A1 N-terminal truncated peptide in a wildtype background have growth defects after bolting, like exo70a1-3 heterozygous plants.
A. EXO70A1 N-terminal expression line has reduced growth rate and small plant architecture.
B and C. Shoot apical region of wildtype (b) and EXO70A1 N-terminal expression line (EXO70A1N T1-36) (c), showing flower development defect and small silique phenotypes in EXO70A1N expression line.
The minimum scale in the rulers in B and C is 1 mm.

FIG. 16 is a panel showing ES2 effects on plant SEC8 and EXO84 and human EXO70 isoforms.
A. ES2 does not affect localization pattern of Arabidopsis SEC8 and EXO84. Localization of SEC8 (left panels) and EXO84 (right panels) in root transition zone after 4 hours of treatment by DMSO (top panels) or ES2 (bottom panels).
B. ES2 affects localization of human EXO70 proteins. Localization of GFP-tagged isoform2 (left panels) and isoform5 (right panels) of human EXO70 in Hela cells treated with DMSO (top panels) or ES2 (bottom panels).
Bars=10 μm

FIG. 17 is a panel showing the STD-NMR spectrum of EXO70A1 wild-type protein and EXO70A1-L596A;I613A under the same ES2 and protein concentrations.
A and B. The spectrum of STD-NMR using 10 μM purified EXO70A1 protein (A) or 10 μM purified EXO70A1-L596A;I613A (B) and 400 μM ES2. The off resonance, on resonance and different spectra are shown.
C. Alignment of EXO70 proteins C-terminus sequence around ES2 binding site. The two asterisks (*) indicate amino acids L596 and I613 that are conserved between Arabidopsis EXO70A1 and EXO70 proteins in human and mouse. Bars on second line indicate relative conservation at amino acid position.

FIG. 18 is an ¹H NMR Spectrum of 4-Amino-3-fluoro-N′[(E)-(4-hydroxy-3-iodo-5-phenyl)methylidene]benzohydrazide (DMSO-d₆, 400 MHz, 298 K).

FIG. 19 is a ¹³C NMR Spectrum of 4-Amino-3-fluoro-N′[(E)-(4-hydroxy-3-iodo-5-phenyl)methylidene]benzohydrazide (DMSO-d₆, 100 MHz, 298 K).

FIG. 20 is an ¹H NMR Spectrum of 4-Amino-3-fluorophenylhydrazide (DMSO-d₆, 400 MHz, 298 K).

FIG. 21 is a ¹³C NMR Spectrum of 4-Amino-3-fluorophenylhydrazide (DMSO-d₆, 100 MHz, 298 K).

FIG. 22 is an ¹⁹F NMR Spectrum of 4-Amino-3-fluorophenylhydrazide (DMSO-d₆, 376 MHz, 298 K).

FIG. 23 is an ¹H NMR Spectrum of 4-Amino-N′-[(E)-(4-hydroxy-3-methoxyphenyl)methylidene]benzohydrazide (DMSO-d₆, 400 MHz, 298 K).

FIG. 24 is a ¹³C NMR Spectrum of 4-Amino-N′-[(E)-(4-hydroxy-3-methoxyphenyl)methylidene]benzohydrazide (DMSO-d₆, 100 MHz, 298 K).

FIG. 25 is an ¹H NMR Spectrum of 4-Aminobenzhydrazide (DMSO-d₆, 400 MHz, 298 K).

FIG. 26 is a ¹³C NMR Spectrum of 4-Aminobenzhydrazide (DMSO-d₆, 100 MHz, 298 K).

FIG. 27 is an ¹H NMR Spectrum of N-(4-((E)-2-(4-hydroxy-3-methoxybenzylidene)hydrazinecarbonyl)phenyl)-5-((3 aS,4S,6aR)-2-oxohexahydro-1H-thieno[3,4-d]imidazol-4-yl)pentanamide (DMSO-d₆, 400 MHz, 298 K).

FIG. 28 is a ¹³C NMR Spectrum of N-(4-((E)-2-(4-hydroxy-3-methoxybenzylidene)hydrazinecarbonyl)phenyl)-5-((3 aS,4S,6aR)-2-oxohexahydro-1H-thieno[3,4-d]imidazol-4-yl)pentanamide (DMSO-d₆, 100 MHz, 298 K).

FIG. 29 is a ¹³C NMR Spectrum of N-(2-fluoro-4-((E)-2-(4-hydroxy-3-iodo-5-methoxybenzylidene)hydrazinecarbonyl)phenyl)-5-((3 aS,45,6aR)-2-oxohexahydro-1H-thieno[3,4-d]imidazol-4-yl)pentanamide (DMSO-d₆, 100 MHz, 298 K).

FIG. 30 is an ¹H NMR Spectrum of N-(2-fluoro-4-((E)-2-(4-hydroxy-3-iodo-5-methoxybenzylidene)hydrazinecarbonyl)phenyl)-5-((3 aS,45,6aR)-2-oxohexahydro-1H-thieno[3,4-d]imidazol-4-yl)pentanamide (DMSO-d₆, 400 MHz, 298 K).

FIG. 31 is an ¹H NMR Spectrum of N-(2-fluoro-4-((E)-2-(4-hydroxy-3-iodo-5-methoxybenzylidene)hydrazinecarbonyl)phenyl)-5-((3 aS,45,6aR)-2-oxohexahydro-1H-thieno[3,4-d]imidazol-4-yl)pentanamide (DMSO-d₆, 100 MHz, 298 K).

FIG. 32 is Table 6 listing various peptides identified in pull-down assays.

### DETAILED DESCRIPTION

The chemical structure of ES2 is shown in FIG. 1A. Analogs of ES2 can include, for example, a substituted or non-substituted iodine-containing phenyl group of ES2 and/or a substituted or non-substituted fluorine-containing benzoic ring of ES2.

The term “substituted” refers to a hydrocarbyl group (including an aryl group) in which one or more bonds to a hydrogen atom contained within the group is replaced by a bond to a non-hydrogen atom of a substituent group. Examples of non-hydrogen atoms include, but are not limited to, carbon, oxygen, nitrogen, phosphorus, sulfur, selenium, arsenic, chlorine, bromine, silicone and fluoride. Examples of substituent groups include halo, perhaloalkyl such as trifluoromethyl, hydroxy, amino, alkoxy, aryloxy, carboxy, mercapto, cyano, nitro, ester, ether, thioether, trialkylsilyl, amide and hydrocarbyl group.

In some embodiments, the N′-benzylidenebenzohydrazide analog of compound ES2 can be a heteroatom-containing analog. The term “heteroatom-containing” refers to a molecule or molecular fragment (such as the iodine-containing phenyl group or the fluorine-containing benzoic ring of ES2) in which one or more carbon atoms is replaced with an atom other than carbon, such as but not limited to, nitrogen, oxygen, sulfur, phosphorus or silicon. For example, in some embodiments of the N′-benzylidenebenzohydrazide analog of compound ES2, the iodine-containing phenyl group or the fluorine-containing benzoic ring, or both, can each independently be substituted and heteroatom-containing.

Modifying exocyst complex activity can include promoting or inhibiting exocyst complex activity. Inhibiting exocyst complex activity can include, but is not limited to, inhibiting exocytosis and/or inhibiting endocytic recycling. Promoting exocyst complex activity can include, but is not limited to, increasing exocytosis and/or increasing endocytic recycling.

Modifying, altering, increasing or decreasing exocytosis or endocytic recycling in a cell is relative to exocytosis or endocytic recycling in a control cell. Typically, a control cell is not exposed to a test compound.

In some embodiments, a plant, fungal or animal cell is exposed to a test compound such as ES2 or an ES2 analog. Examples of plants include, but are not limited to, Arabidopsis, rice, corn, wheat, barley, rye, soybean, tomato, sorghum, cotton, citrus, canola, rape seed, mint, grapes, and turf grasses. Examples of animals include, but are not limited to, humans and other mammals, such as dogs, mice, rats, bovine, cats, sheep, and horse. Examples of fungi include, but are not limited to, plant and animal pathogens such as yeast, Phytopthora, powdery mildew, Botrytis, ringworm, and Cladiporium.

In embodiments involving screening, chemical libraries and small compound libraries can be screened for active compounds. Screens can be conducted under high throughput screening conditions for efficient testing.

In embodiments involving treatment, diseases for treatment con be diseases associated with altered or aberrant exocytosis and/or endocytic recycling. Examples of such diseases include, but are not limited to, diabetes, cancer, and endocrine diseases, or other secretory diseases such as hypersecretion and hyposecretion diseases of substances such as growth hormone, hyprthyroidism, estrogen, and testosterone. For diabetes, a compound can be used that alters exocytosis and/or endocytic recycling by acting to modulate the cellular uptake or secretion of glucose, or the uptake or secretion of insulin. For cancer, a compound can be used that alters exocytosis and/or endocytic recycling by targeting the exocyst complex or associated proteins. Examples of cancers include, but are not limited to, breast cancer and endocrine tumors.

Some embodiments include a pharmaceutical composition. Although oral administration of a compound is a preferred route of administration, other means of administration such as nasal, topical or rectal administration, or by injection or inhalation, are also contemplated. Depending on the intended mode of administration, the pharmaceutical compositions may be in the form of solid, semi-solid or liquid dosage forms, such as, for example, tablets, suppositories, pills, capsules, powders, liquids, suspensions, ointments or lotions, preferably in unit dosage form suitable for single administration of a precise dosage. The compositions can include an effective amount of the selected compound in combination with a pharmaceutically acceptable carrier and, in addition, may include other pharmaceutical agents such as anti-viral agents, adjuvants, diluents, buffers, and the like. The compounds may thus be administered in dosage formulations containing conventional non-toxic pharmaceutically acceptable carriers, adjuvants and vehicles. The amount of active compound administered will be dependent on the subject being treated, the subject's weight, the manner of administration and the judgment of the prescribing physician.

For solid compositions, conventional nontoxic solid carriers include, for example, pharmaceutical grades of mannitol, lactose, starch, magnesium stearate, sodium saccharin, talc, cellulose, glucose, sucrose, magnesium carbonate, and the like. Liquid pharmaceutically administrable compositions can, for example, be prepared by dissolving, dispersing, etc., an active compound as described herein and optional pharmaceutical adjuvants in an excipient, such as, for example, water, saline, aqueous dextrose, glycerol, ethanol, and the like, to thereby form a solution or suspension. If desired, the pharmaceutical composition to be administered may also contain minor amounts of nontoxic auxiliary substances such as wetting or emulsifying agents, pH buffering agents and the like, for example, sodium acetate, sorbitan mono-laurate, triethanolamine sodium acetate, triethanolamine oleate, etc. Actual methods of preparing such dosage forms are known, or will be apparent, to those skilled in this art. For oral administration, the composition will generally take the form of a tablet or capsule, or may be an aqueous or nonaqueous solution, suspension or syrup. Tablets and capsules for oral use will generally include one or more commonly used carriers such as lactose and corn starch. Lubricating agents, such as magnesium stearate, are also typically added. When liquid suspensions are used, the active agent may be combined with emulsifying and suspending agents. If desired, flavoring, coloring and/or sweetening agents may be added as well. Other optional components for incorporation into an oral formulation herein include, but are not limited to, preservatives, suspending agents, thickening agents, and the like.

In embodiments involving treatment, the subject can be a human or other animals including, but not limited to, mammals such as a dog, mouse, rat, cow, cat, sheep and horse.

Exocyst complex function can be assayed by Brefeldin A (BFA) washout experiment using fluorescence-tagged cargo proteins that constantly undergo exocytosis and recycling (for example PIN2 protein); BFA washout experiment using lipophilic dye such as FM4-64 to label the lipid membranes that undergo exocytosis and recycling; using Fluorescence Recovery After Photobleaching (FRAP) experiment to study the dynamics of fluorescence-tagged cargo proteins that undergo constant exocytosis and recycling; studying the dynamics of cargo proteins that are tagged with photo-convertible fluorescence proteins for example Dendra2 using time-lapse imaging.

Binding of a compound to the exocyst complex or a subunit thereof can be assayed by reduction of exocytosis and recycling, reduced exocyst complex protein dynamics and direct interaction between the subunit proteins and the compound.

For example, binding of ES2 (or another compound) to EXO70 protein (or another exocyst complex component) can be assayed by DARTS (Drug Affinity Responsive Target Stability) assay, STD-NMR (Saturation-Transfer Difference Nuclear Magnetic Resonance), or MST (micro Thermophoresis). In DARTS assay, total cell protein extract is incubated with ES2 and then is digested with different concentrations of protease. The protection of EXO70 protein by ES2 is detected by western blot using and anti-EXO70 antibody. In STD-NMR assay, purified recombinant EXO70 protein is mixed with ES2 and the interaction between is detected by standard STD-NMR assay. In MST assay, purified recombinant EXO70 protein is labeled by red fluorescence dye and then incubated with different concentrations of ES2. The binding is detected using a standard MST device. The binding of ES2 to EXO70 protein can also be assayed by reduced exocytosis of a cargo protein named PIN2 using time-lapse imaging after ES2 treatment, BFA washout of PIN2 in the presence of ES2, microscale thermophoresis, nuclear magnetic resonance, plasmon resonance, and FRAP analysis of EXO70 dynamics.

The exocyst complex subunits are described in UniProtKB/Swiss-Prot database at National Center for Biotechnology Information (NCBI) as follows: in yeast (Saccharomyces cerevisiae): Exocyst complex component EXO70 (Accession: P19658.1); Exocyst complex component EXO84 (Accession: P38261.1); Exocyst complex component SEC10 (Accession: Q06245.1); Exocyst complex component SEC5 (Accession: P89102.1); Exocyst complex component SEC3 (Accession: P33332.1); Exocyst complex component SEC8 (Accession: P32855.1); Exocyst complex component SEC15 (Accession: P22224.2); Exocyst complex component SEC6 (Accession: P32844.2) (all exocyst complex component sequences are incorporated by reference herein). The exocyst complex protein composition and function are conserved between species in different kingdoms such as fungus, animals and plants. Thus, proteins that are homologous to yeast exocyst complex subunits are also subunits of the exocyst complex in corresponding species.

The present invention may be better understood by referring to the accompanying examples, which are intended for illustration purposes only and should not in any sense be construed as limiting the scope of the invention.

### Example 1

### ES2 Inhibits Trafficking to the Plasma Membrane and Enhances Trafficking to the Vacuole

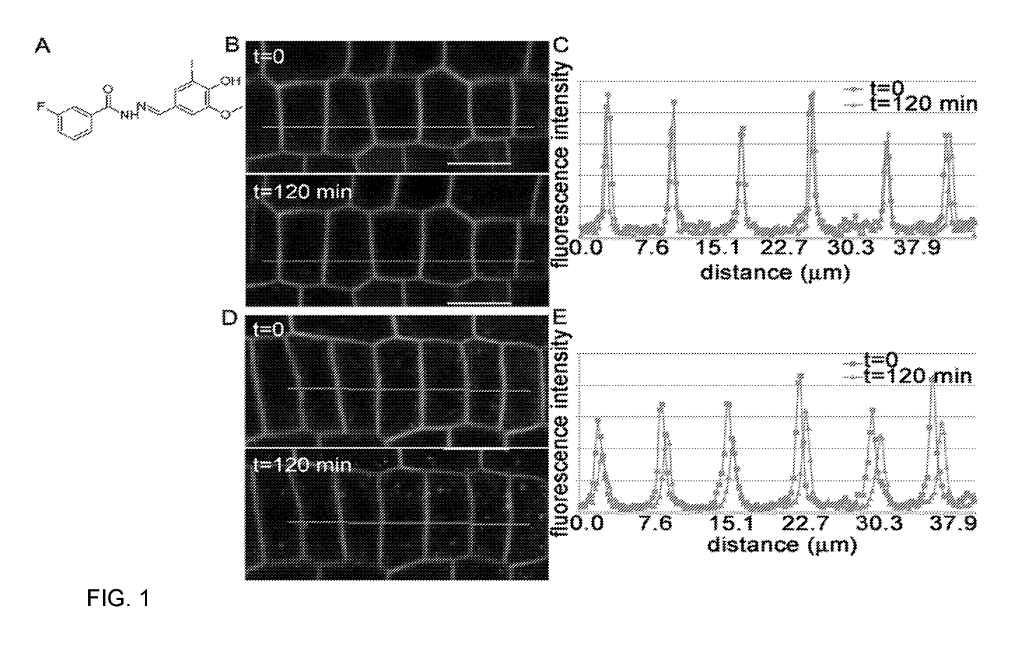

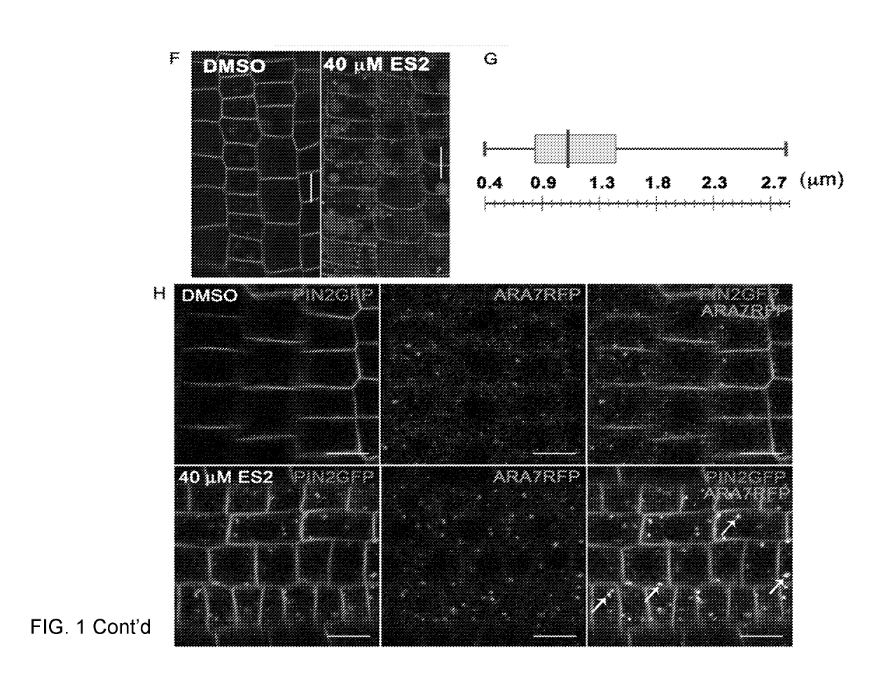

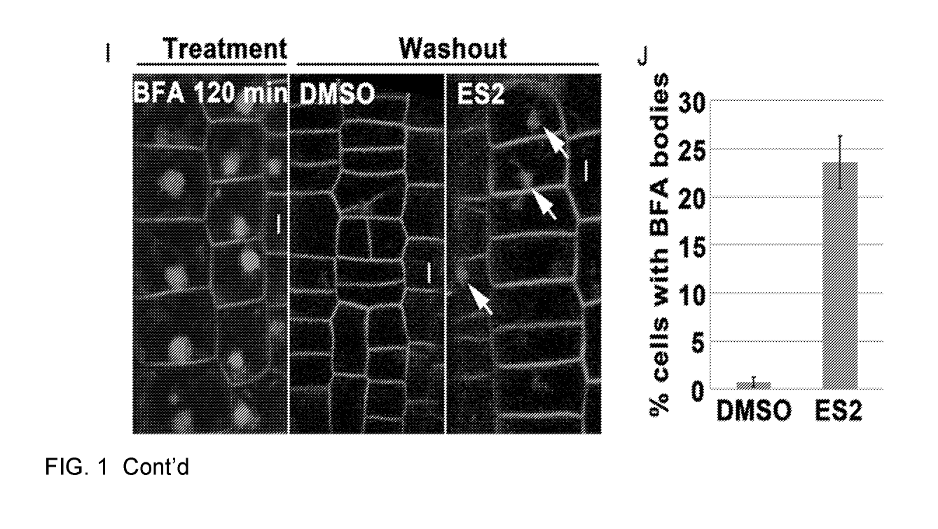

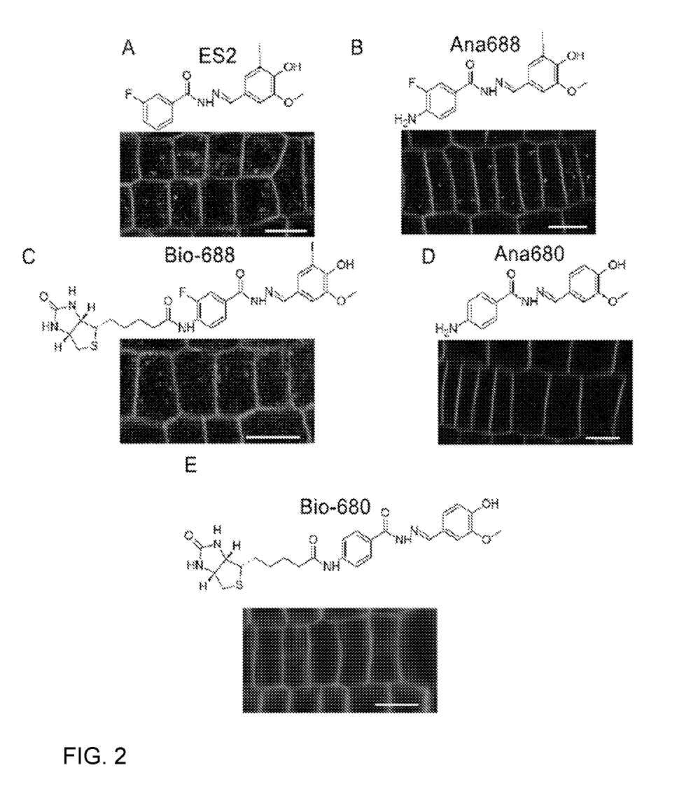

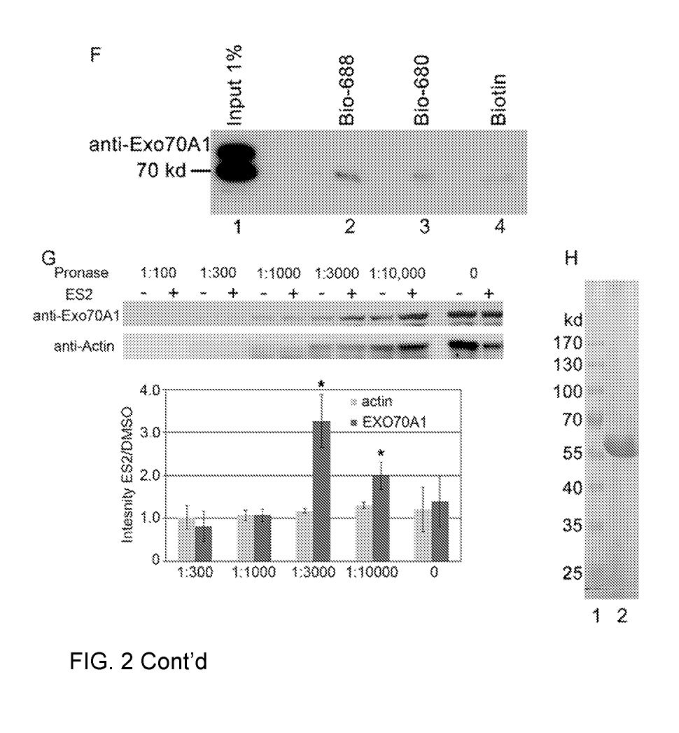

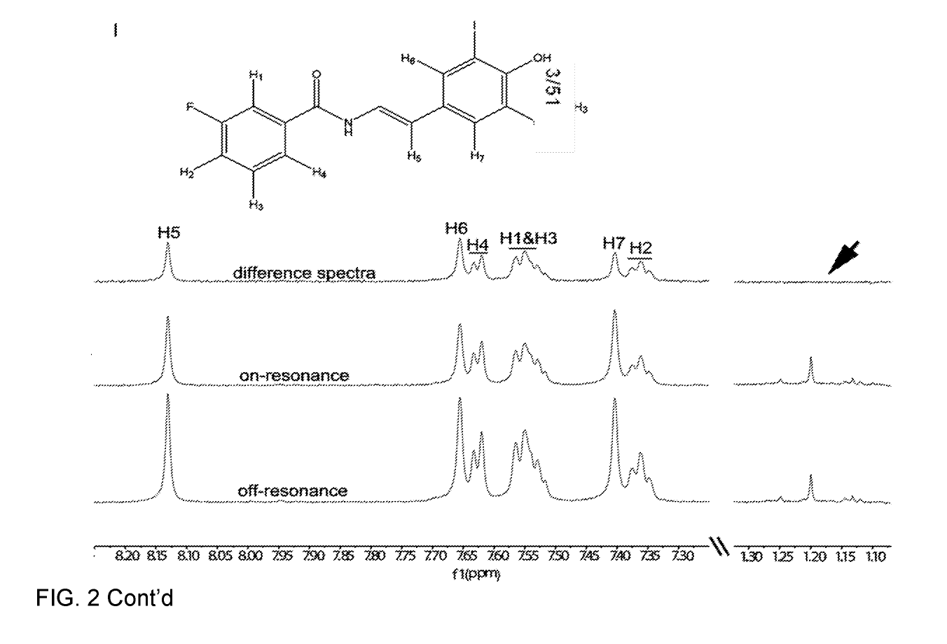

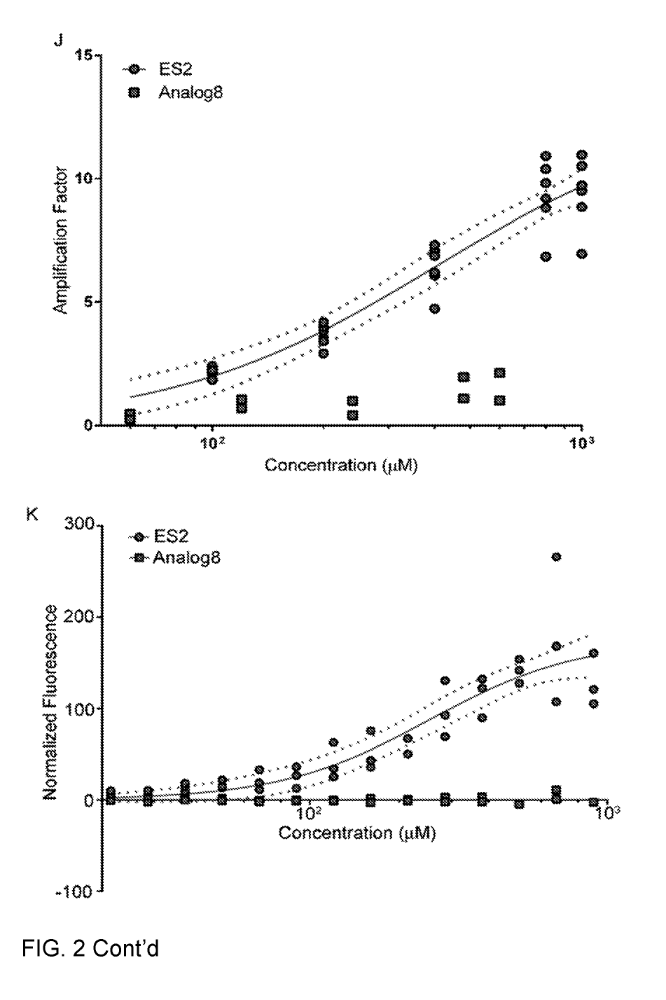

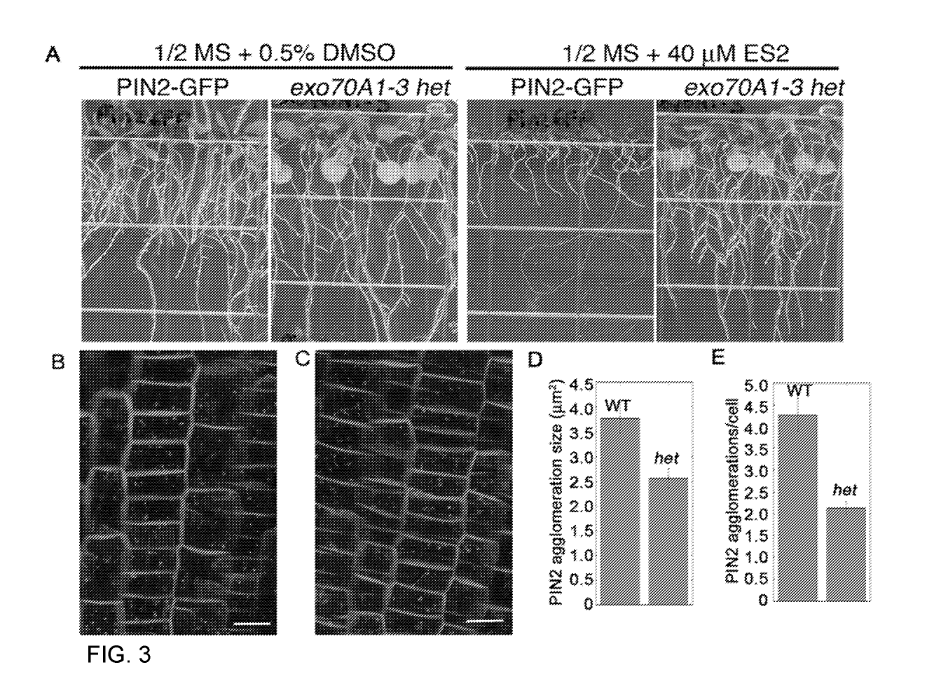

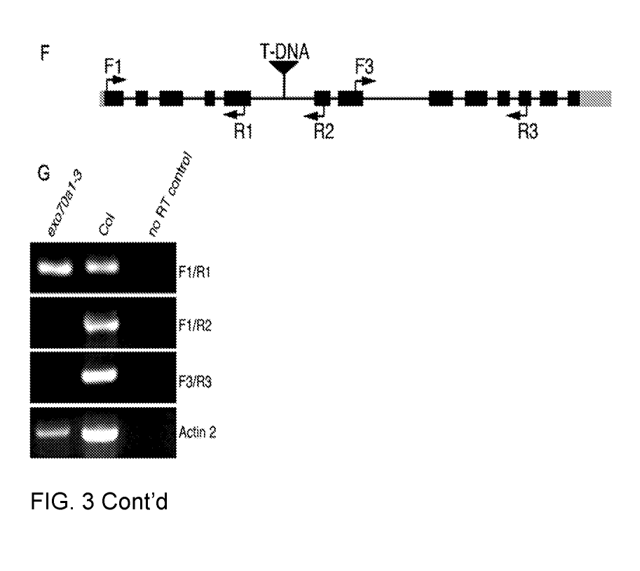

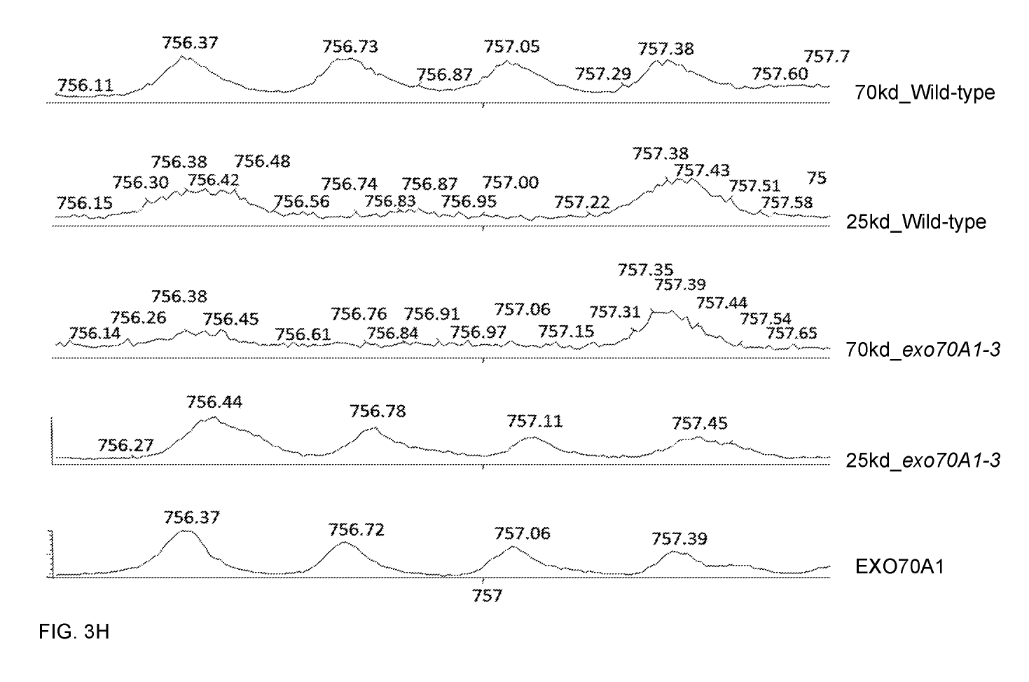

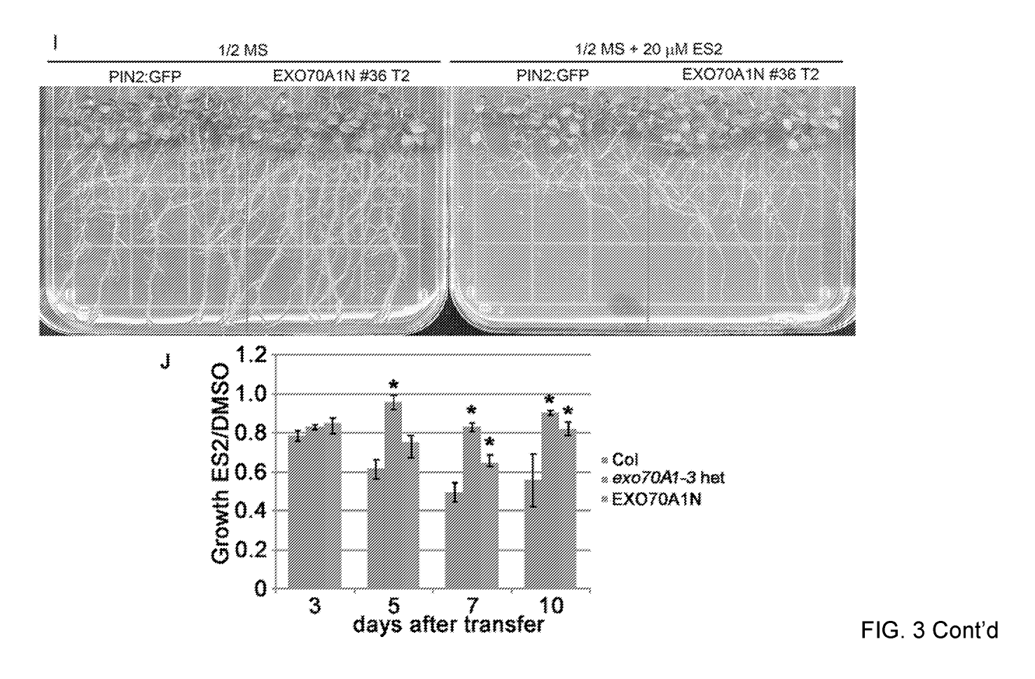

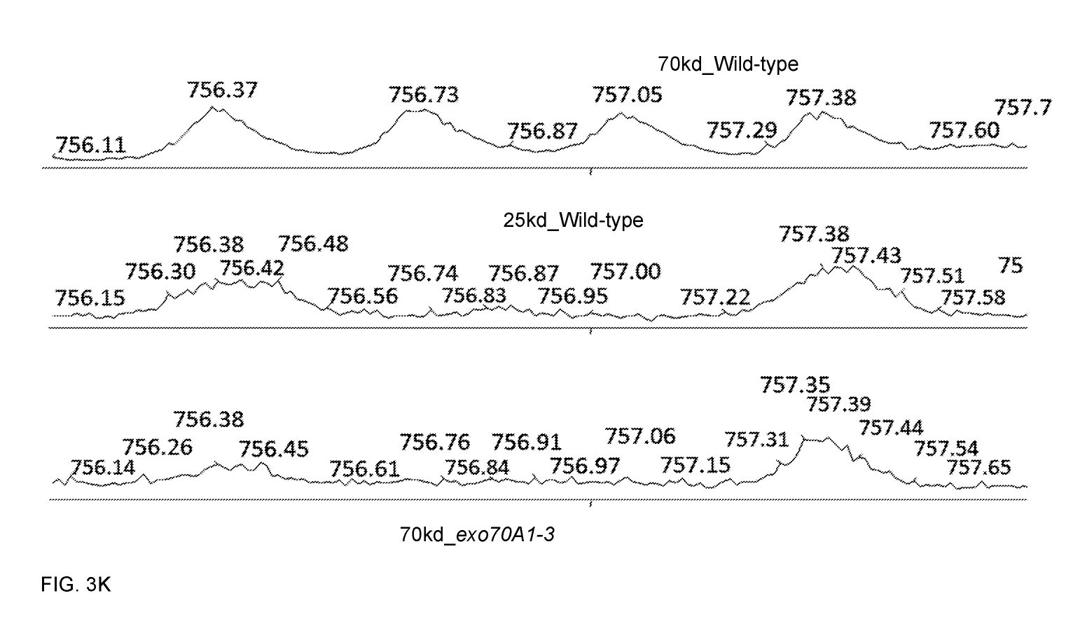

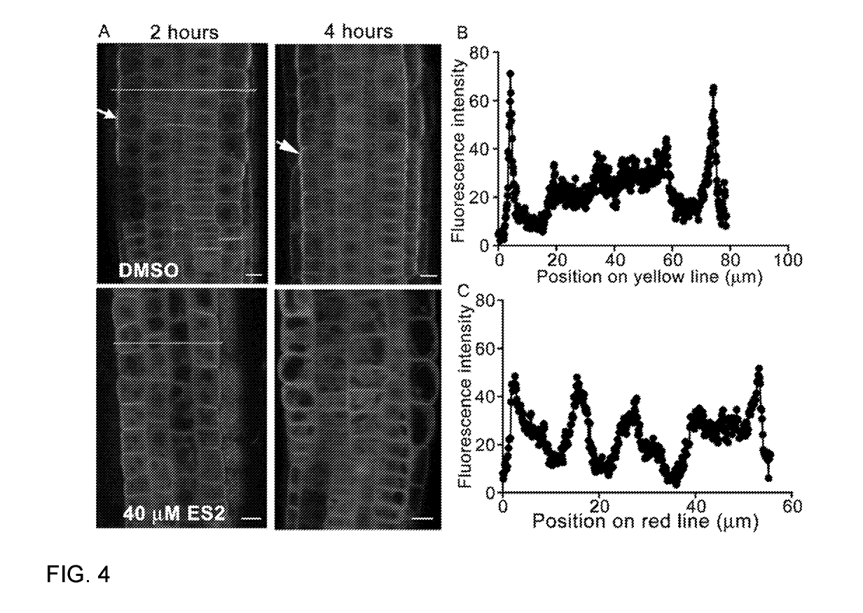

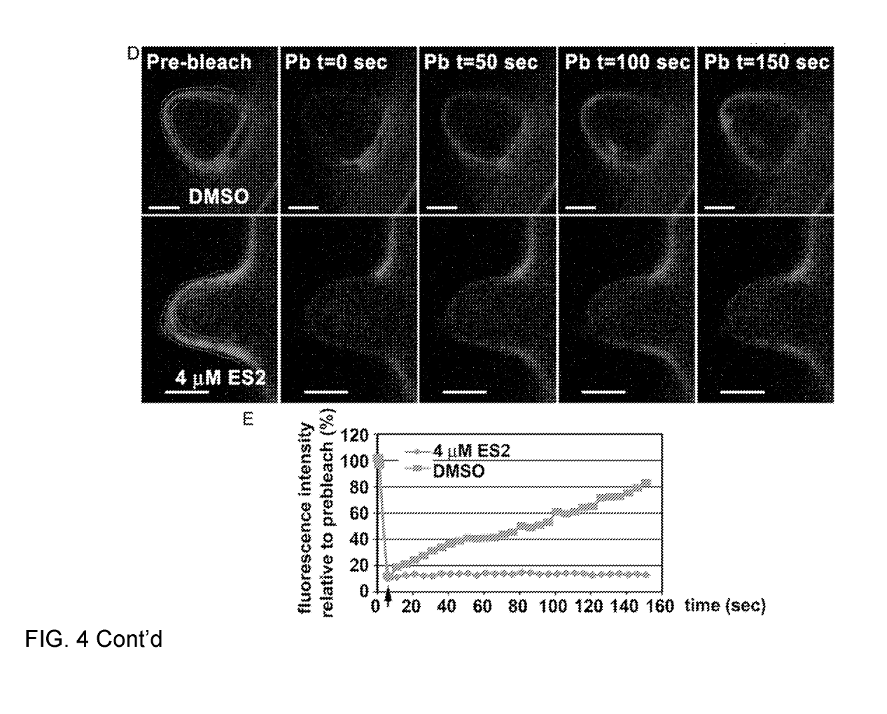

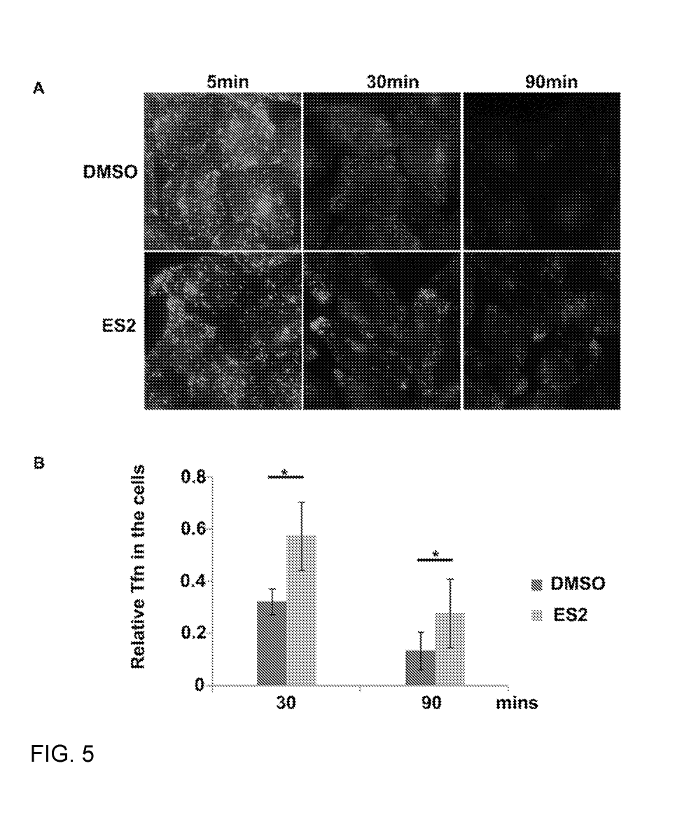

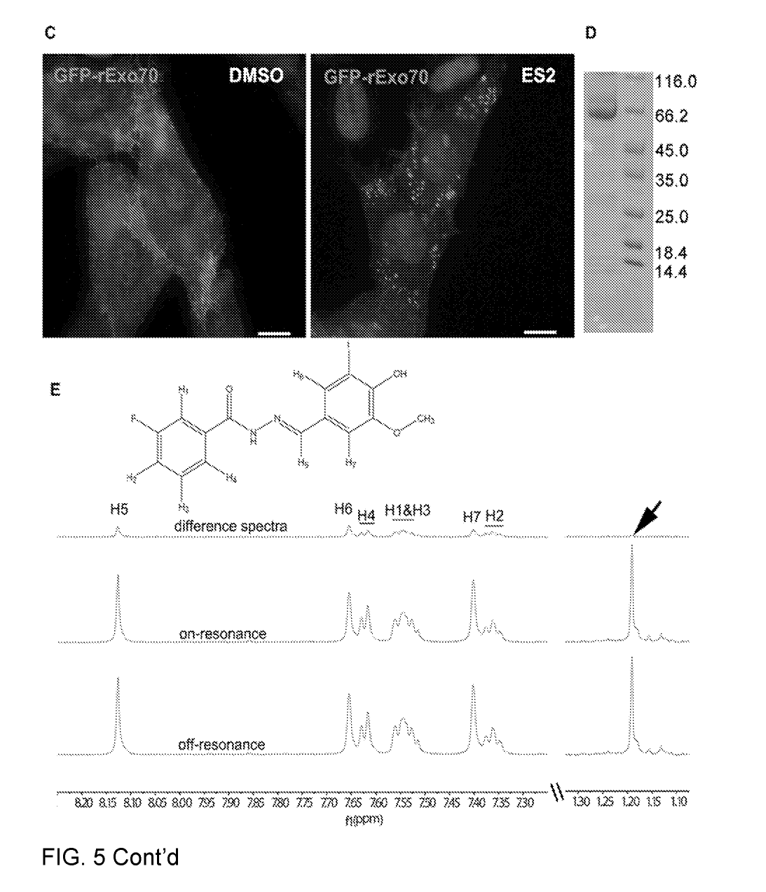

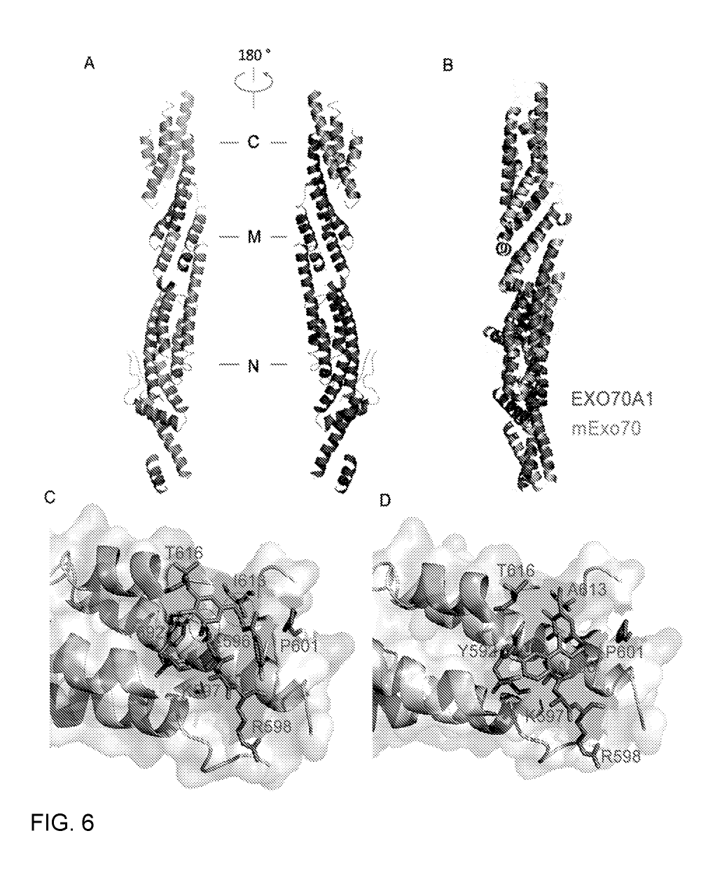

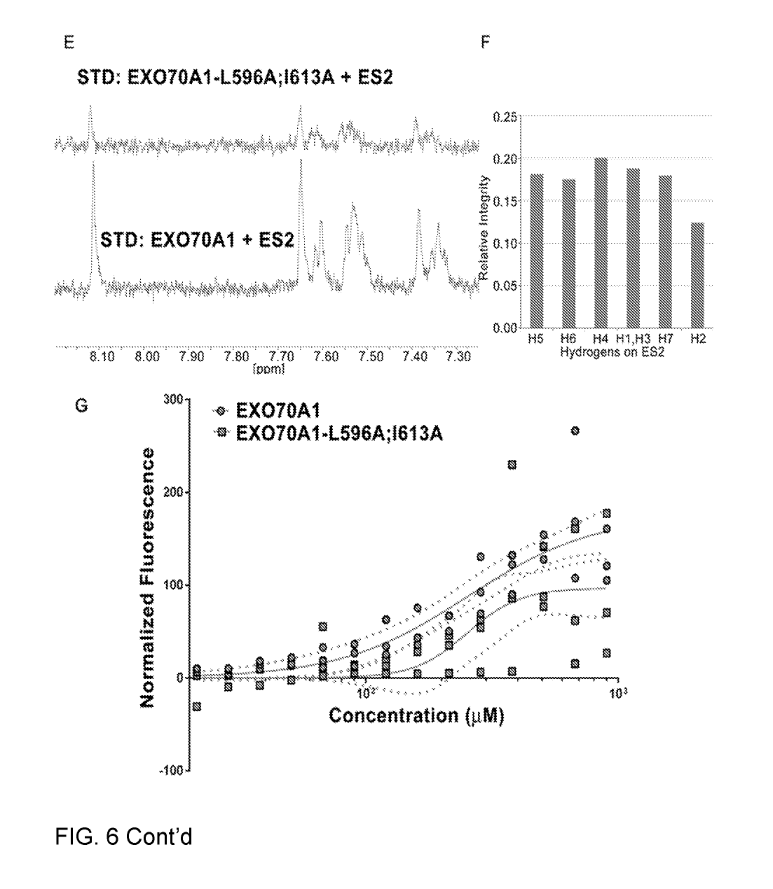

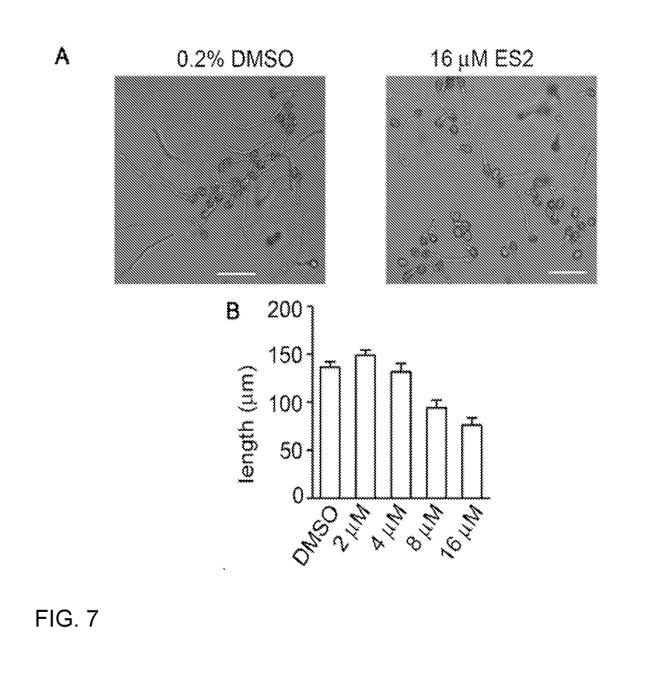

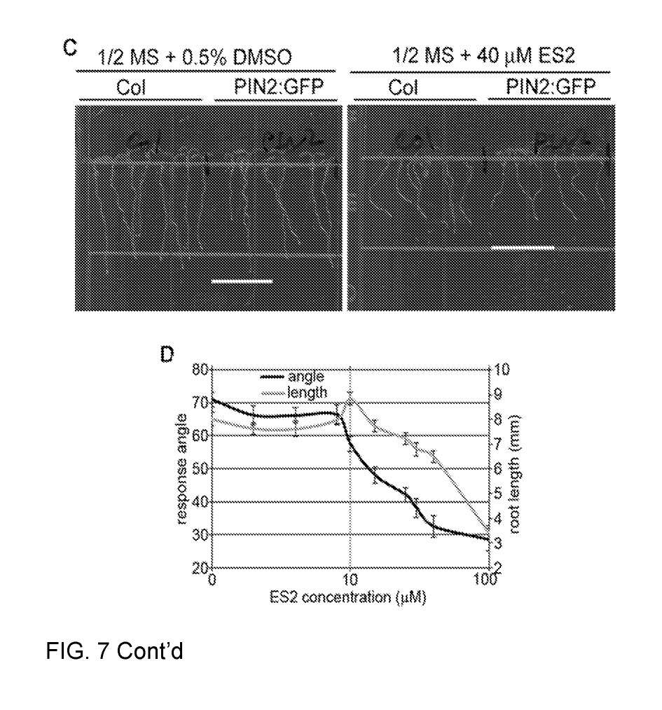

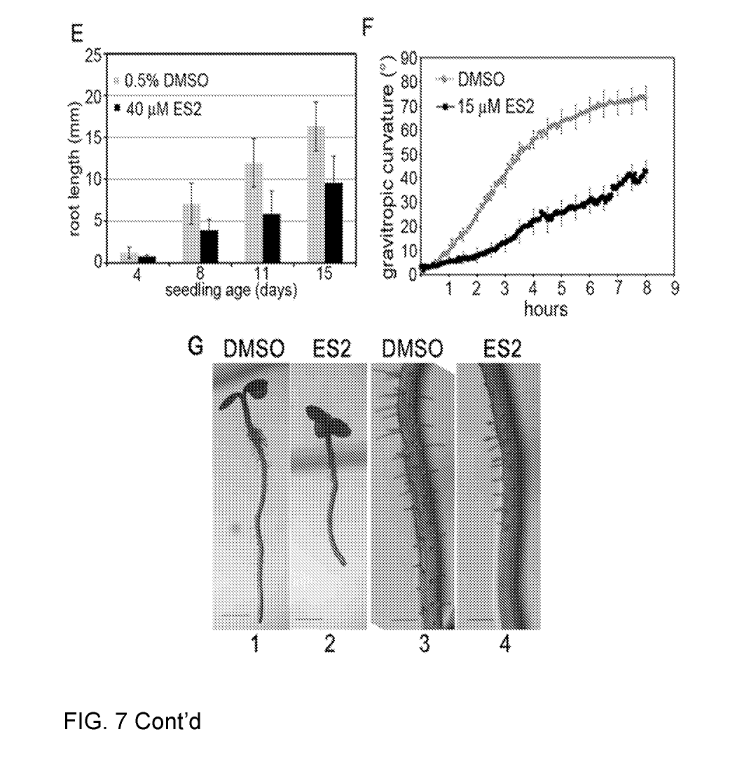

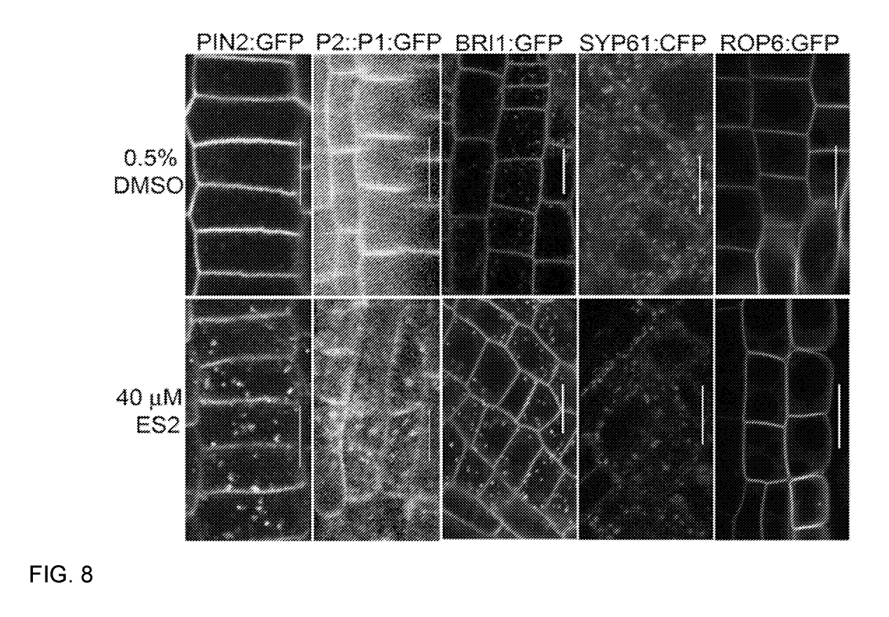

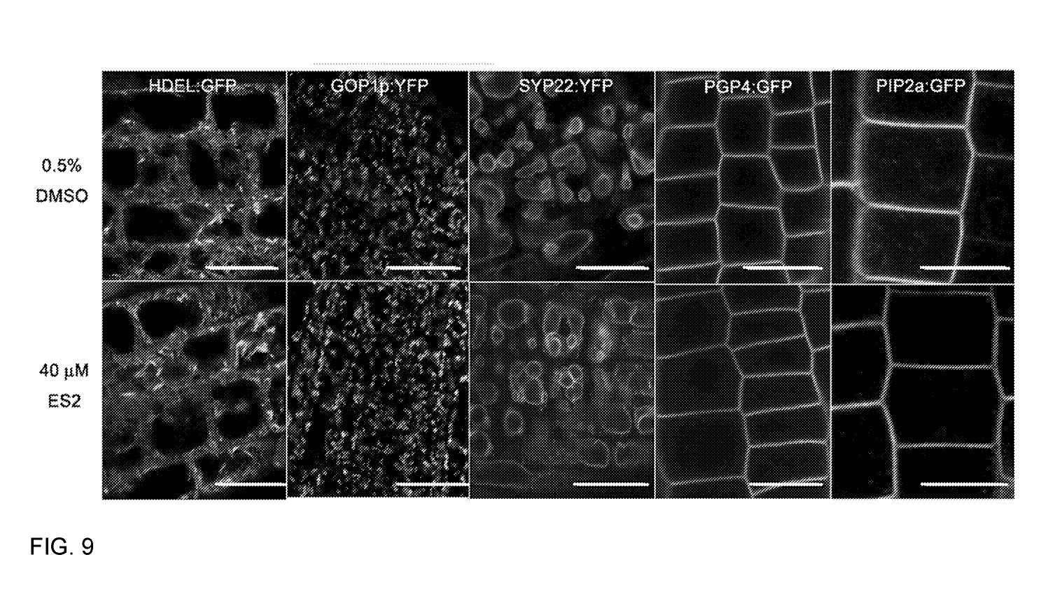

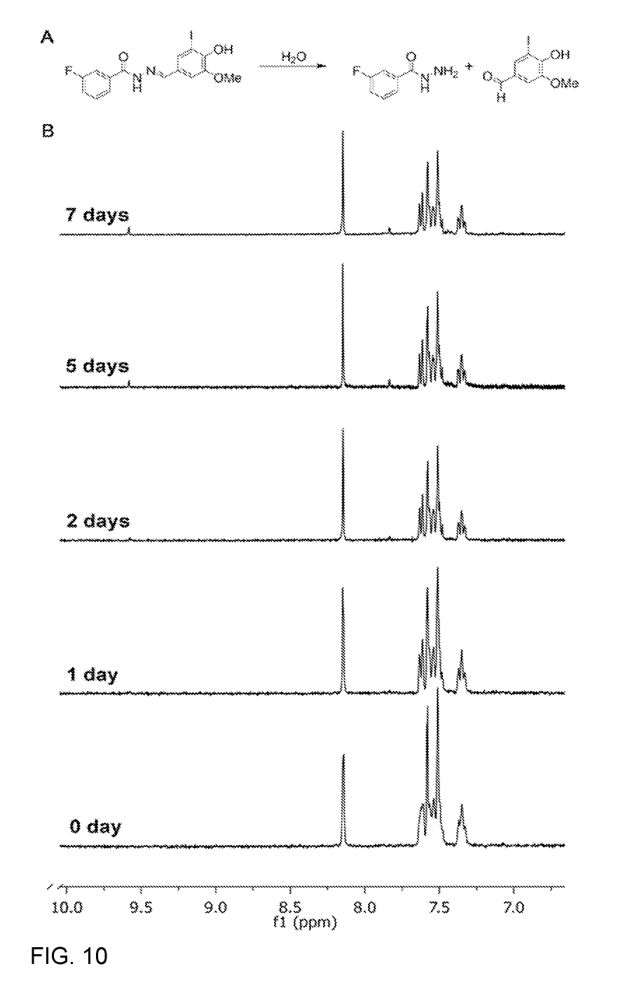

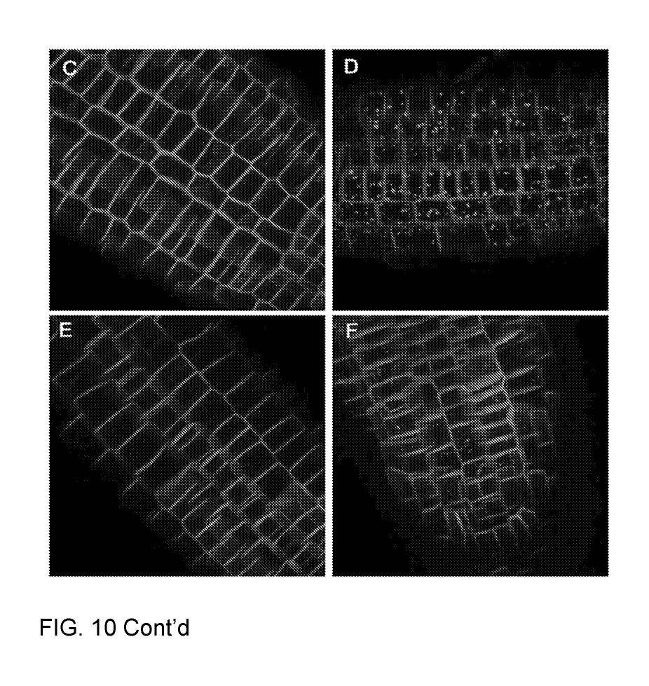

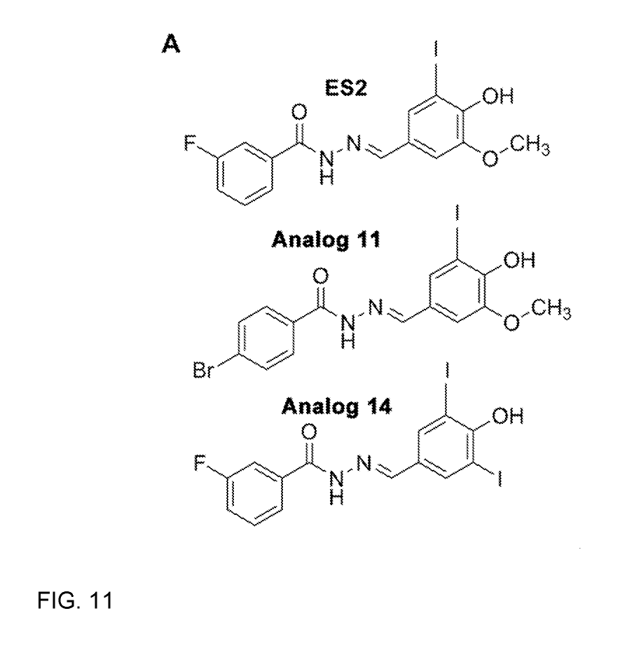

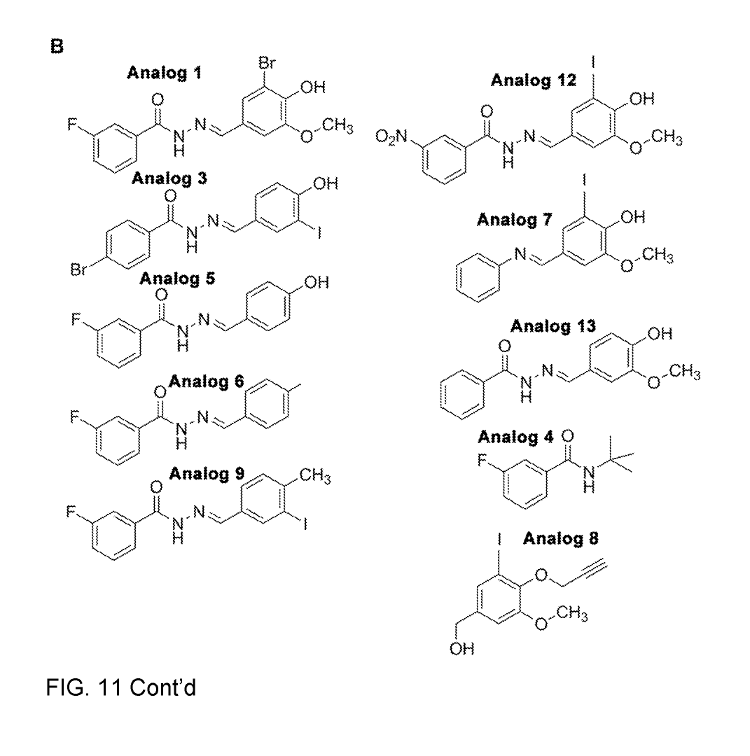

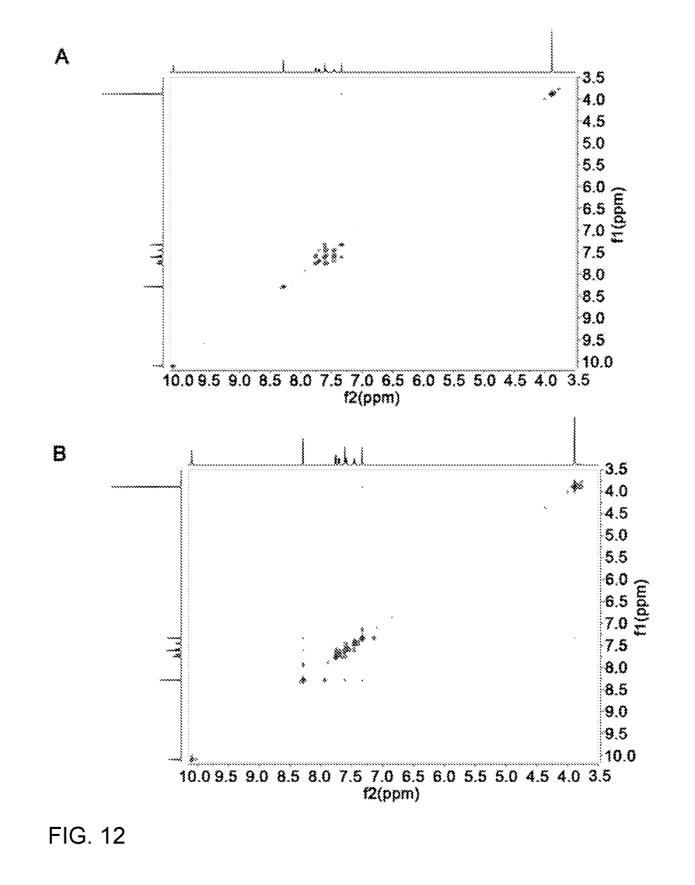

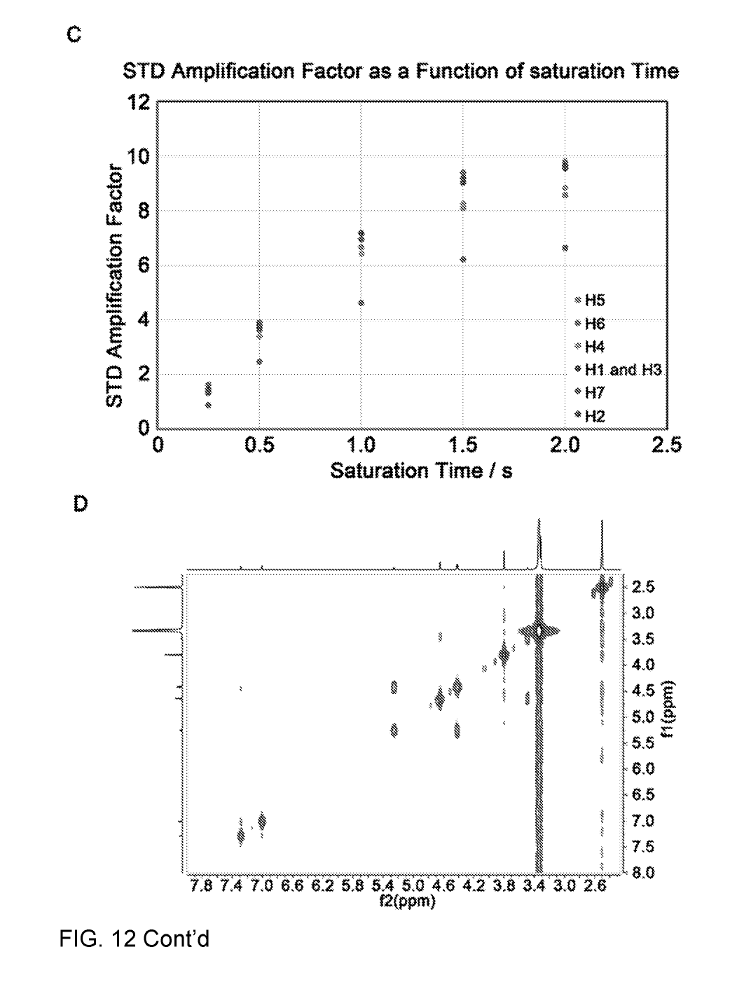

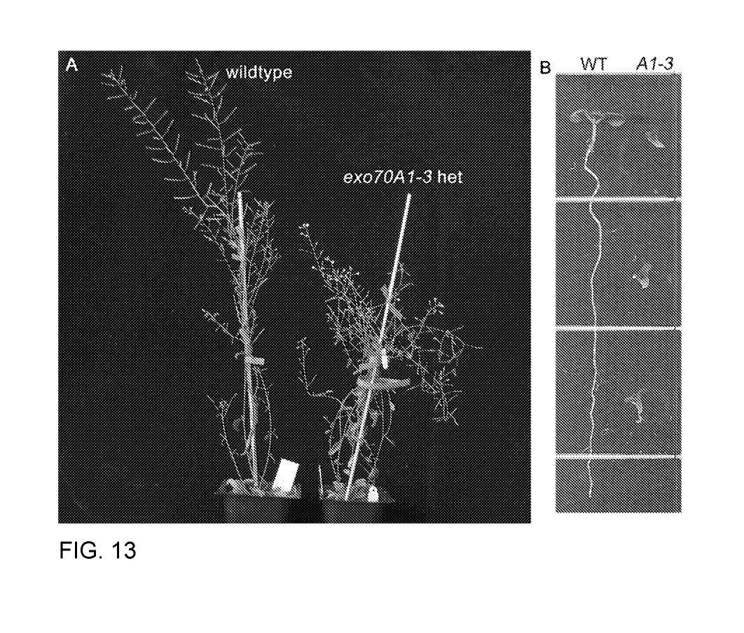

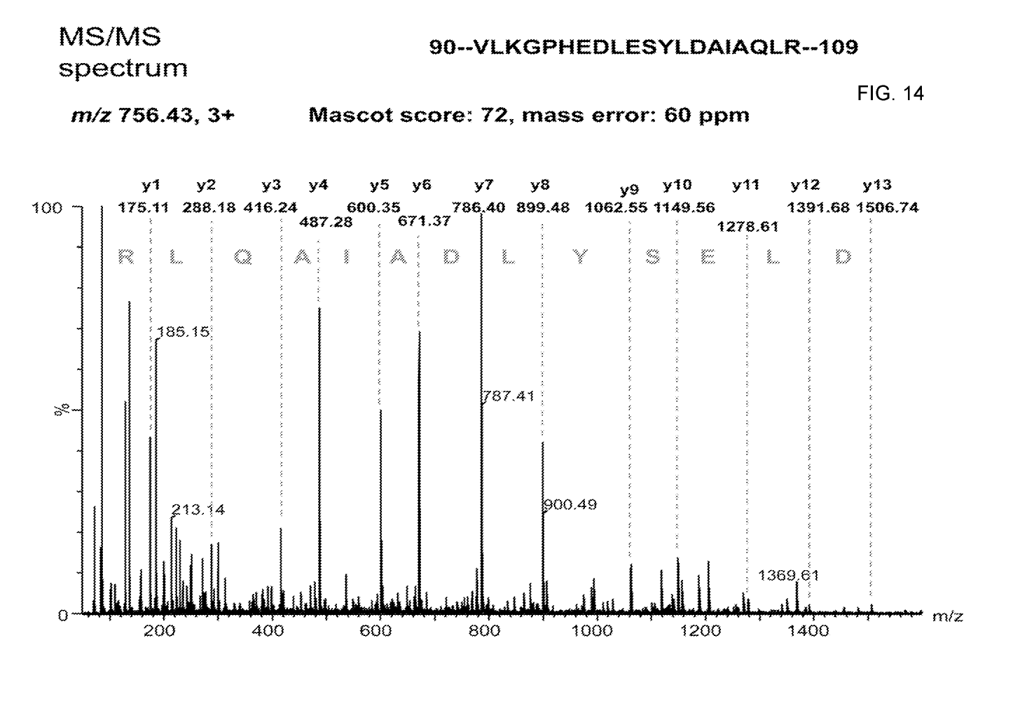

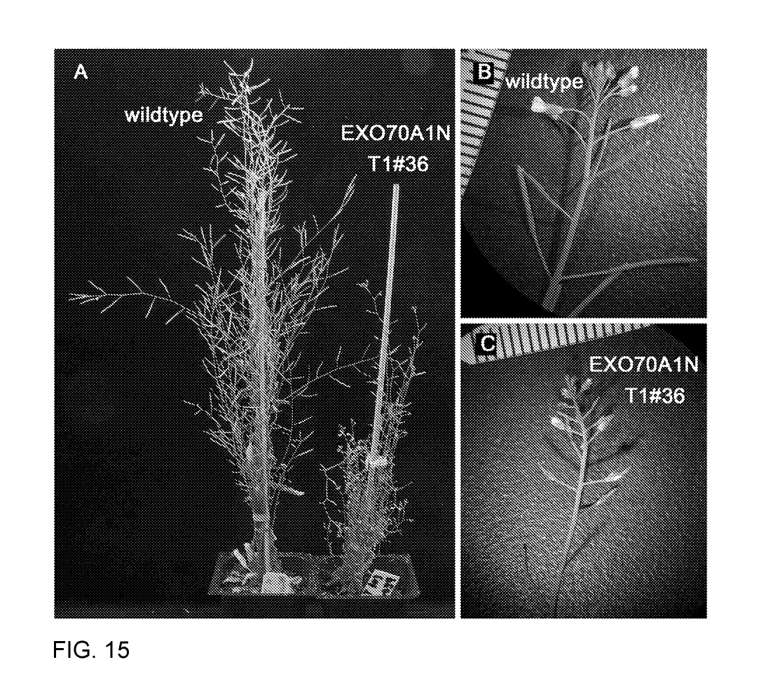

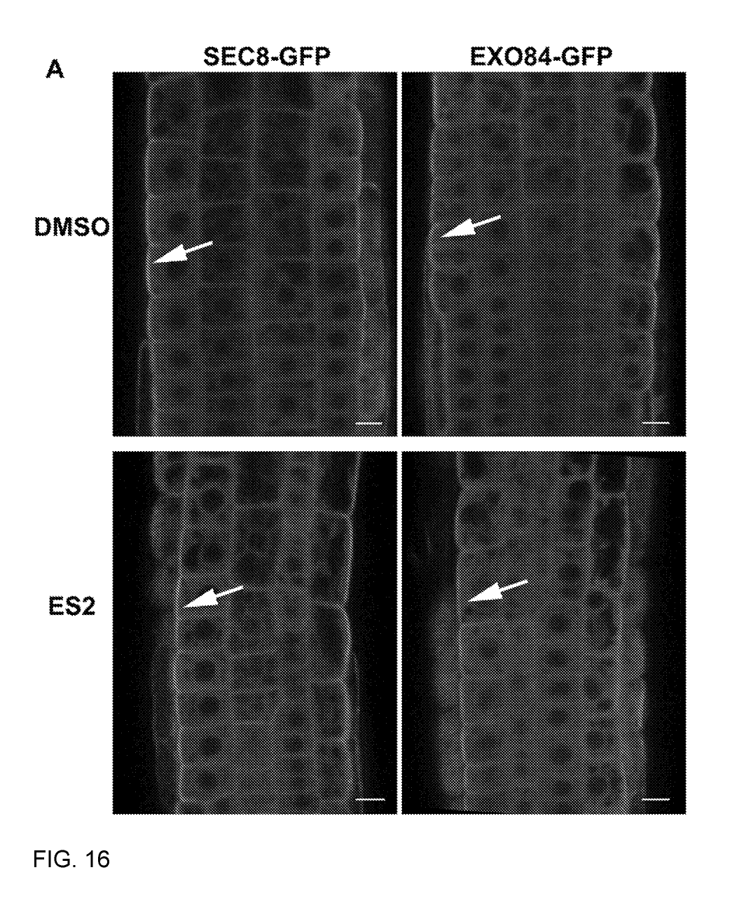

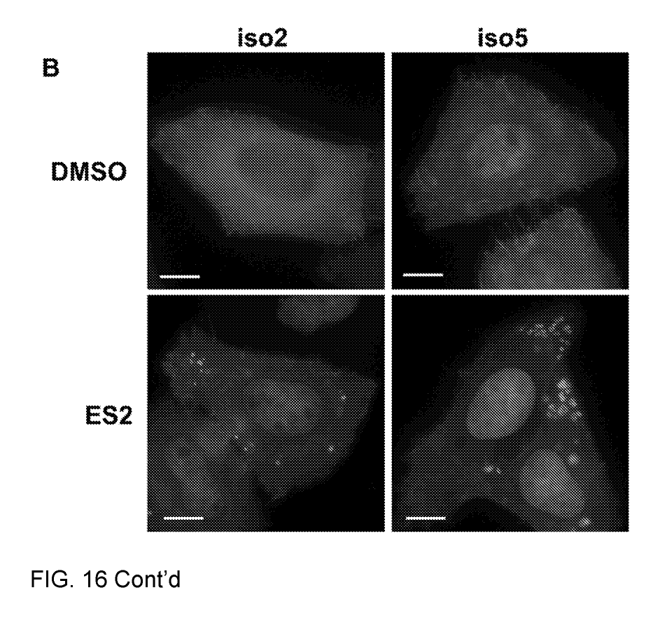

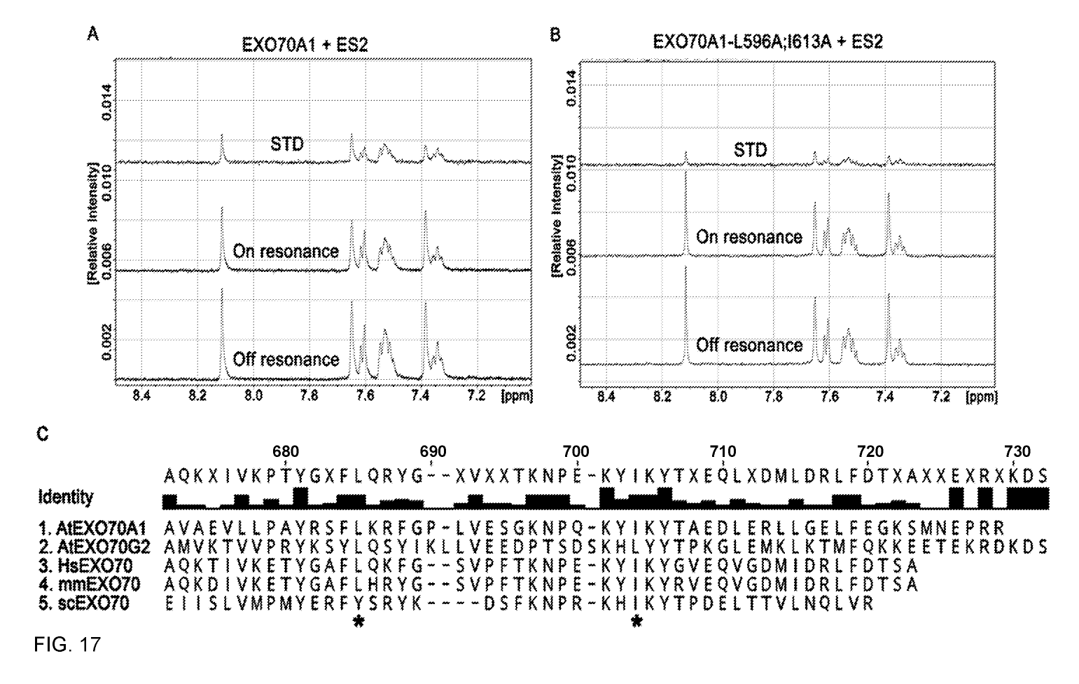

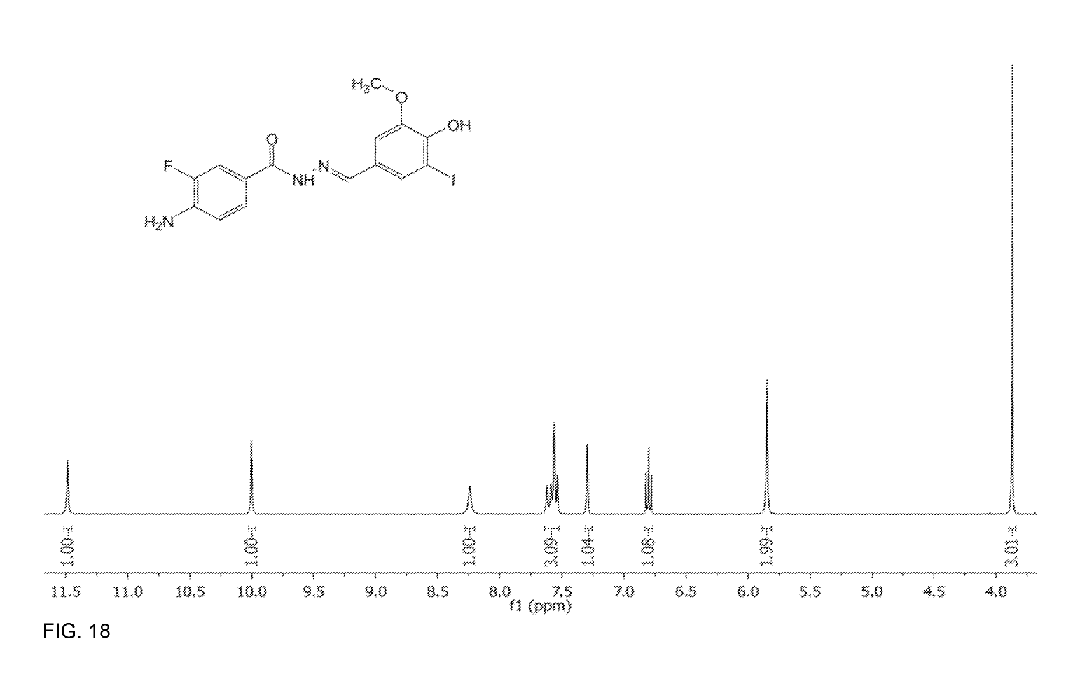

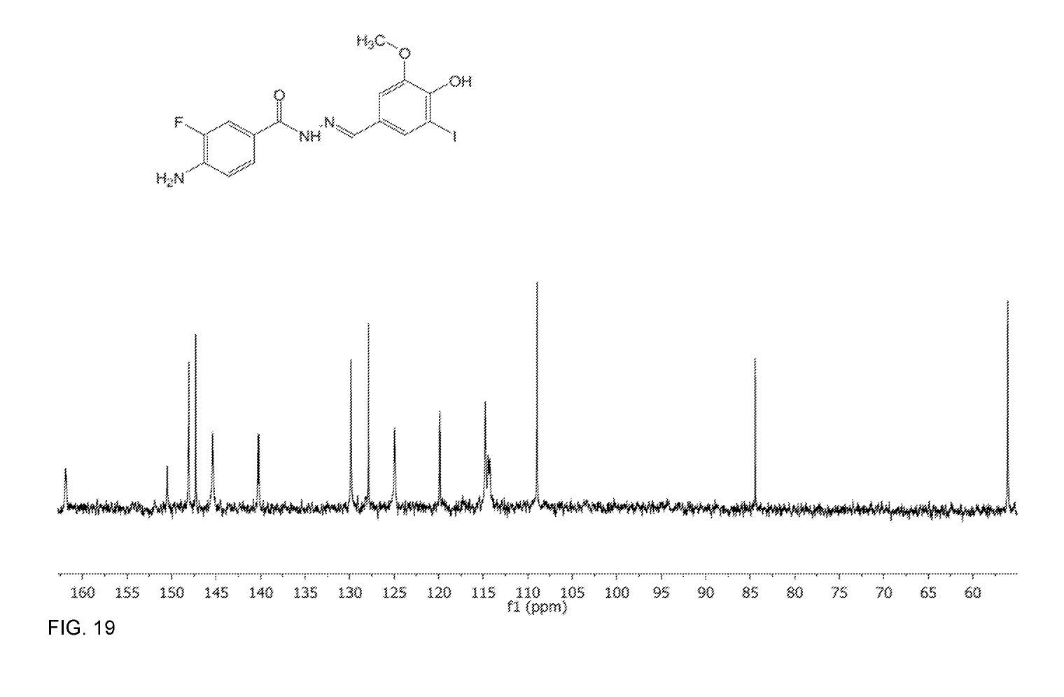

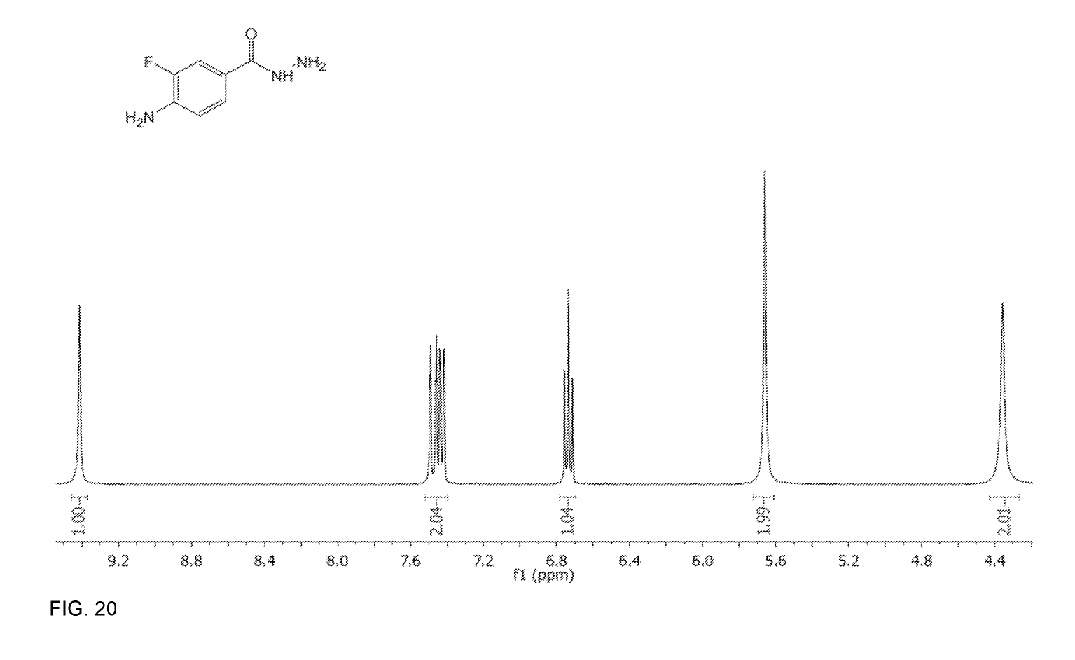

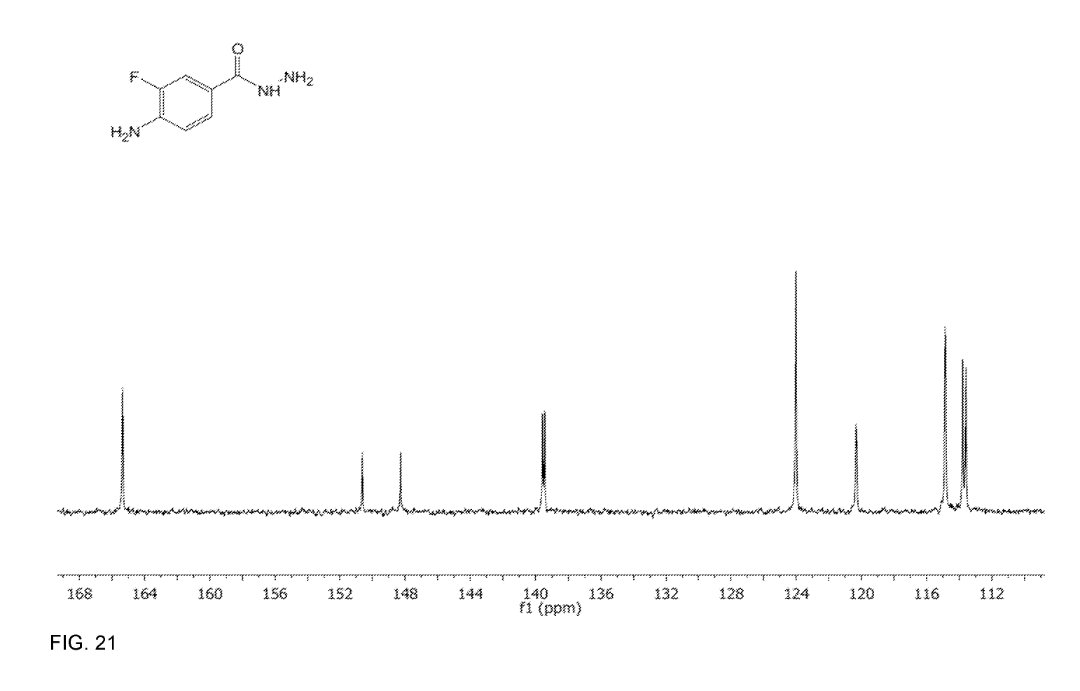

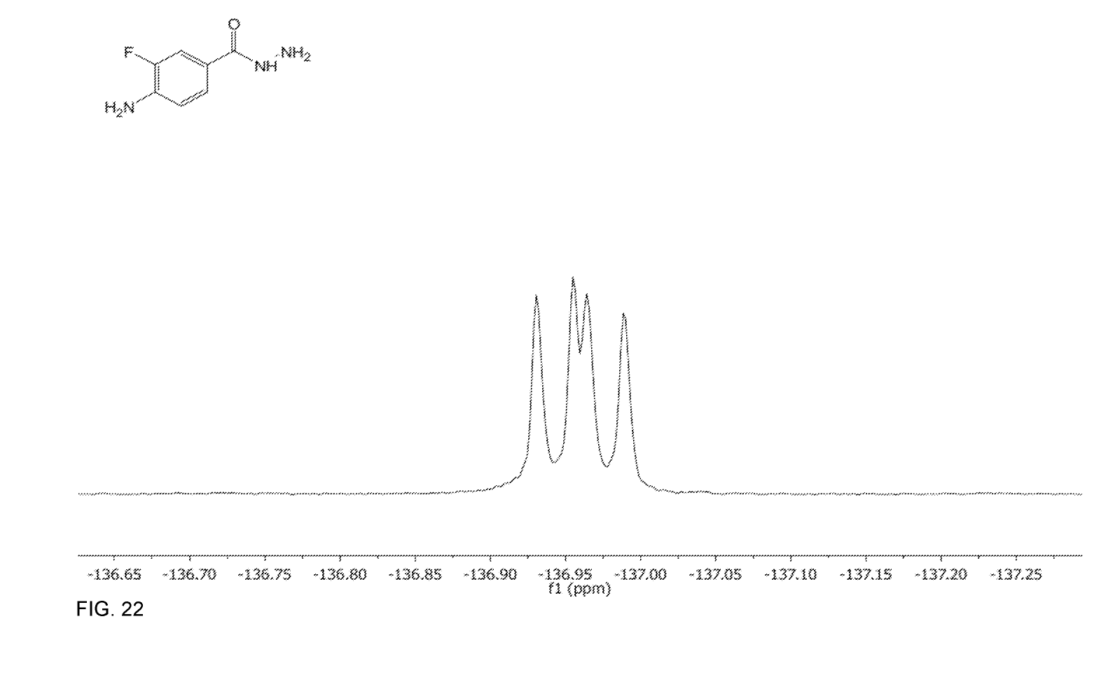

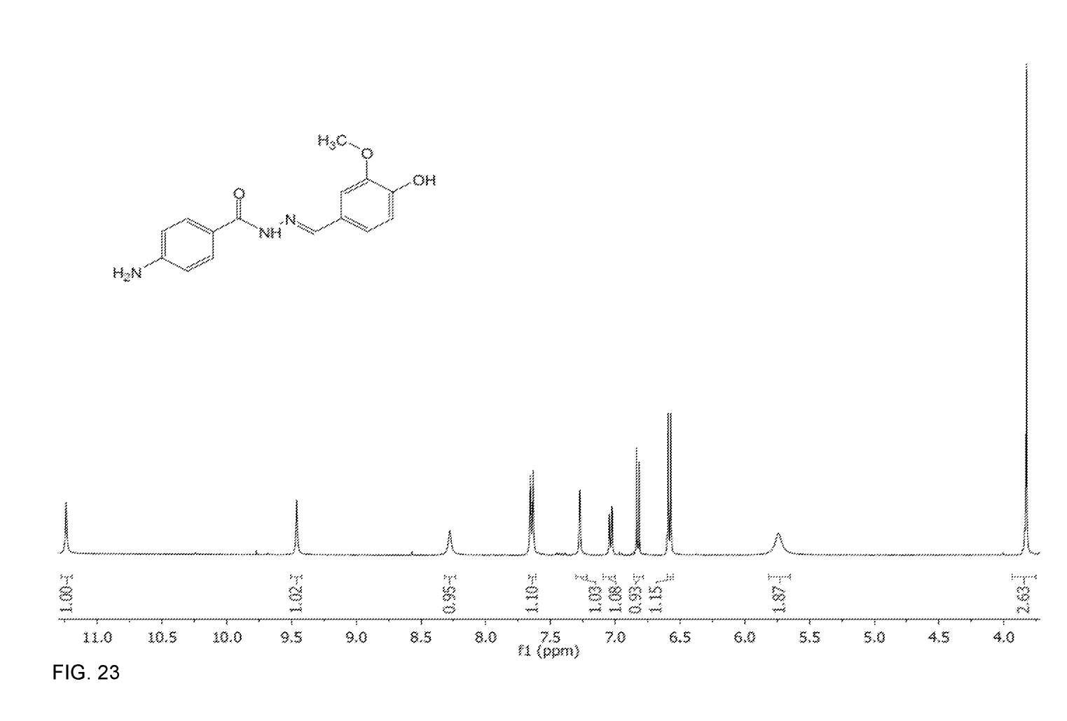

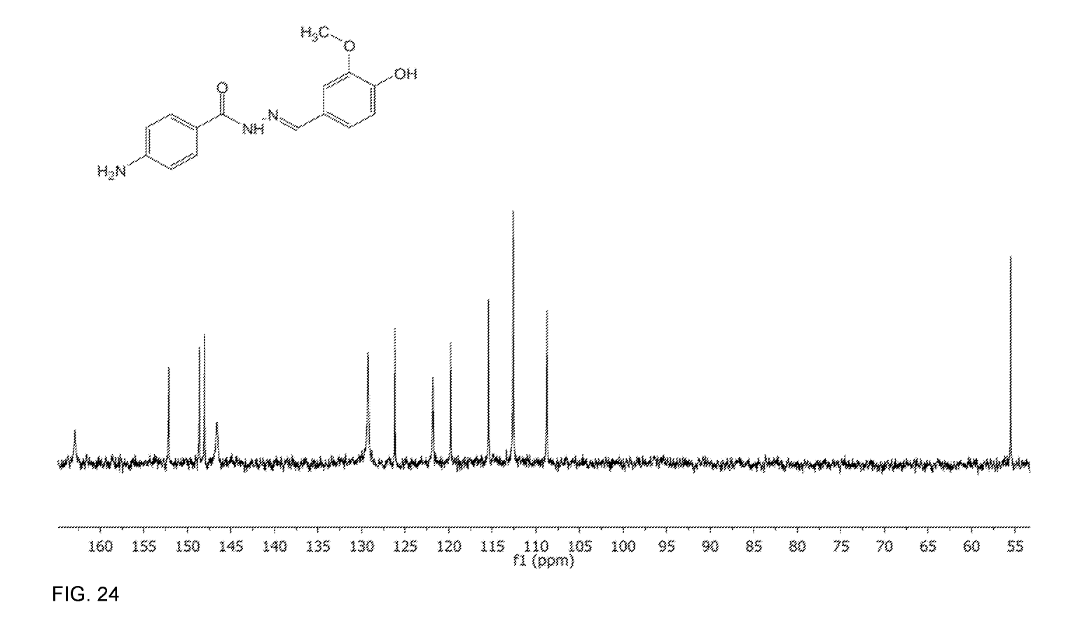

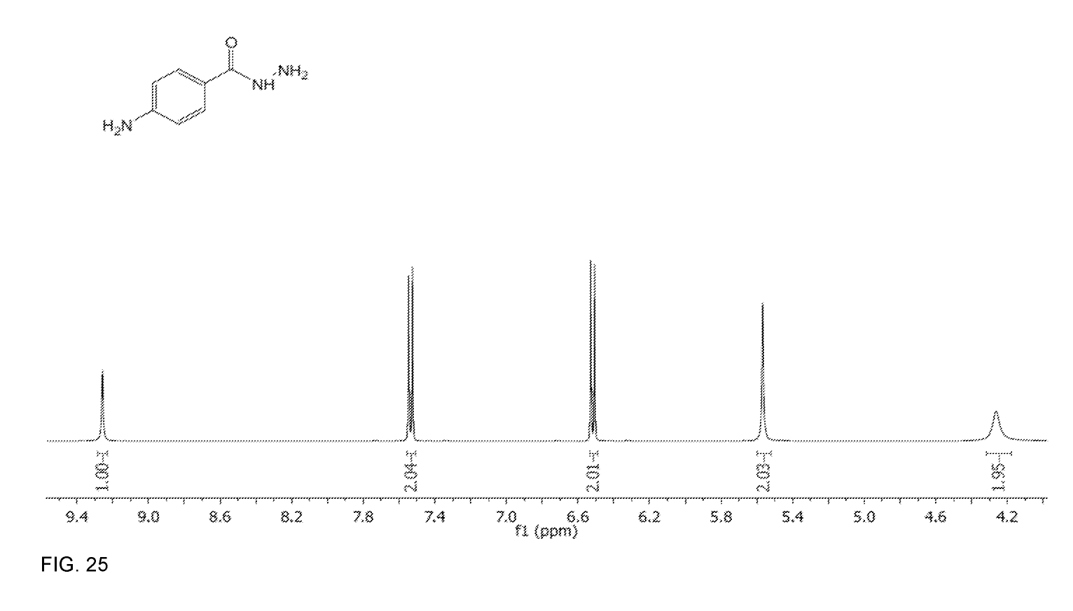

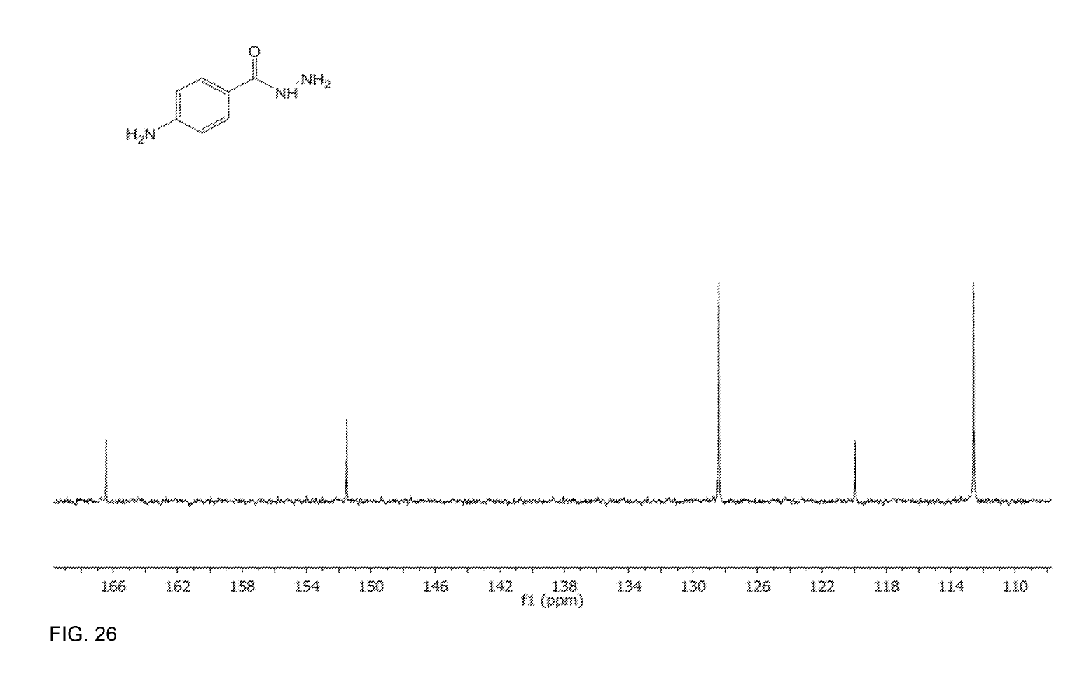

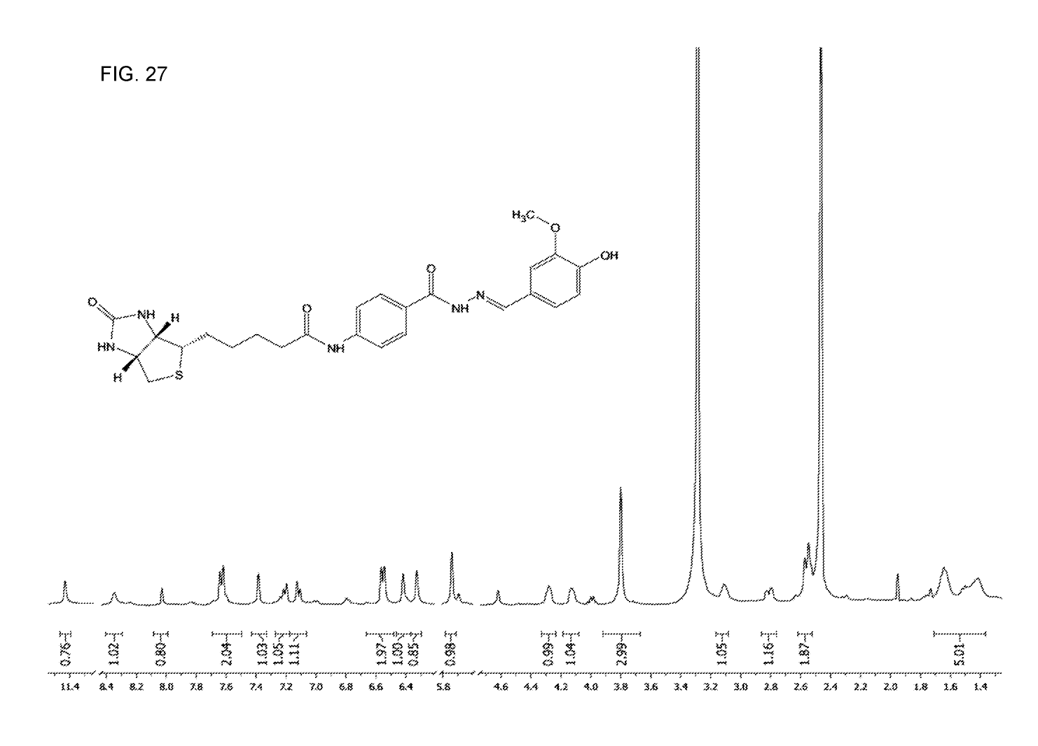

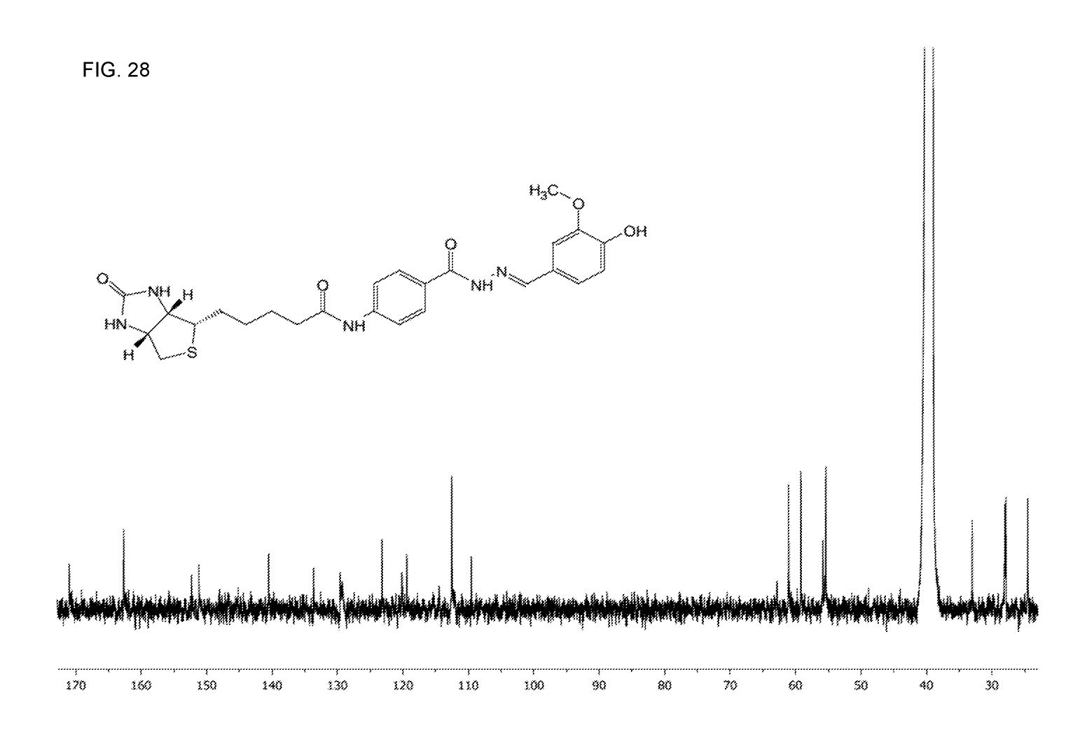

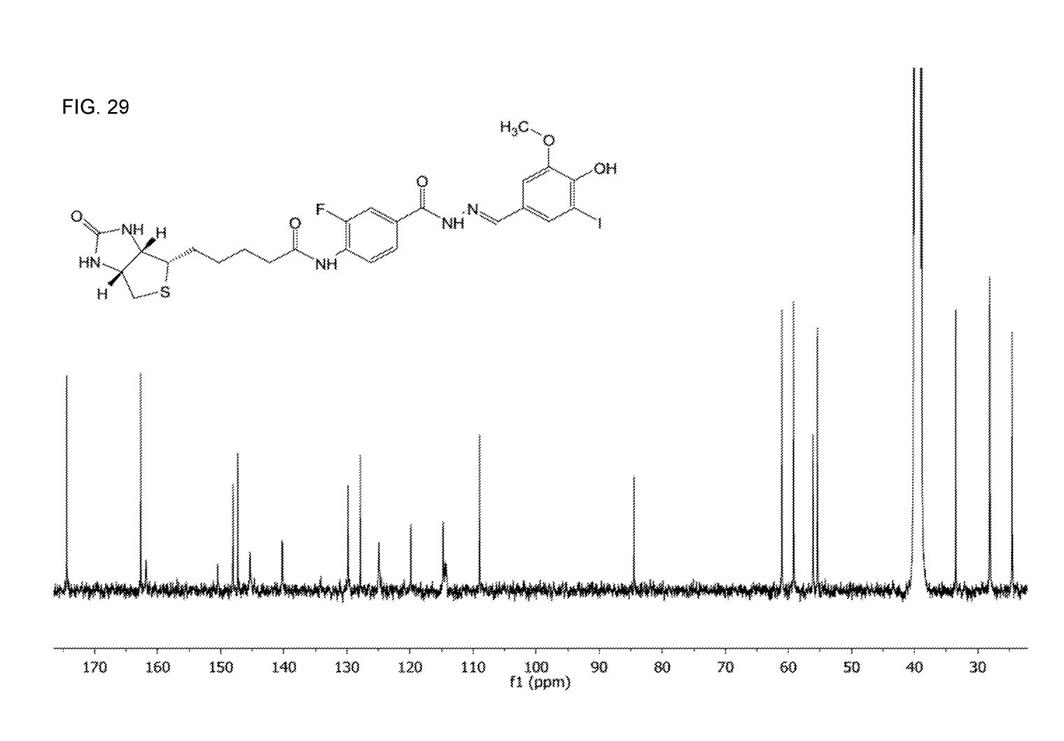

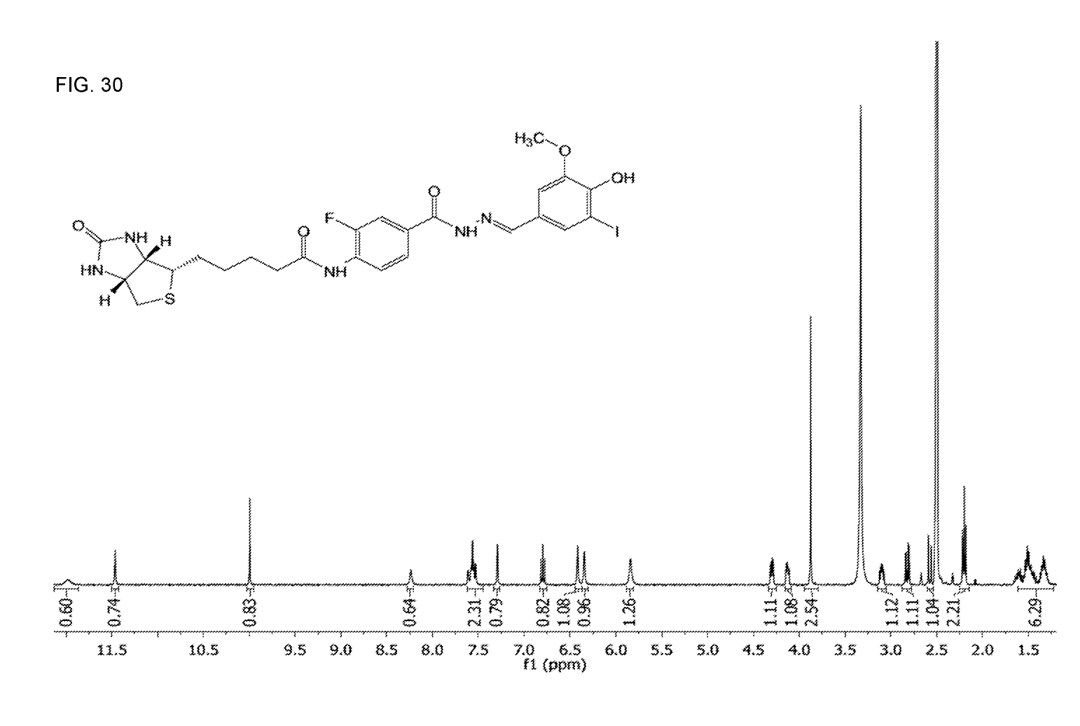

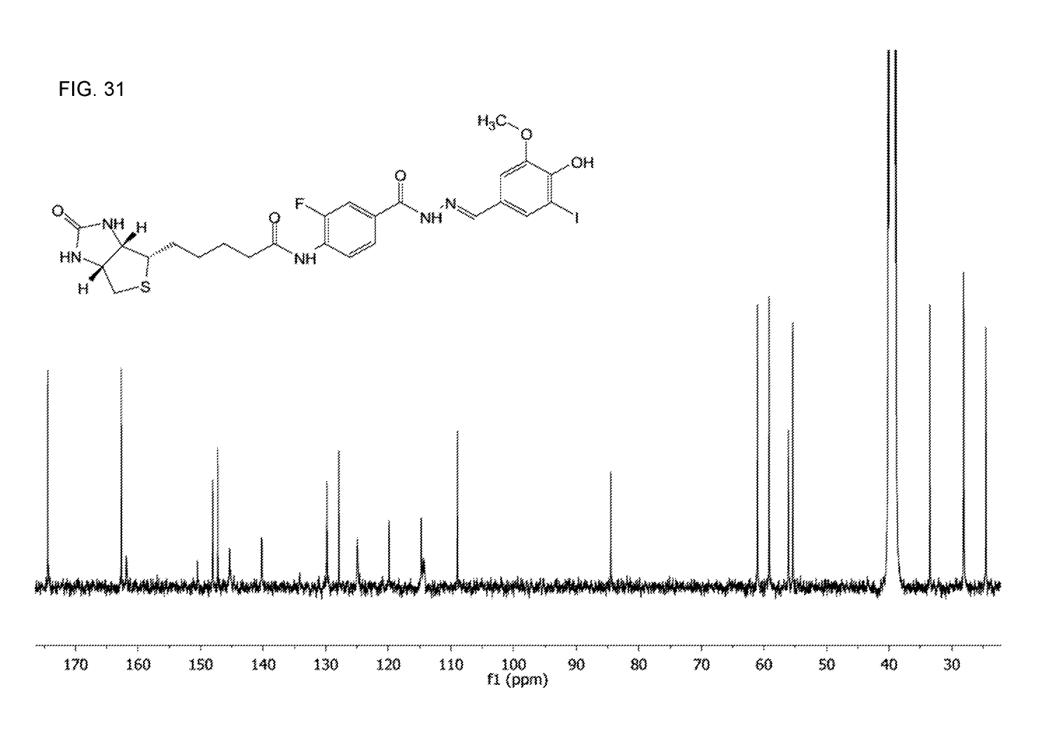

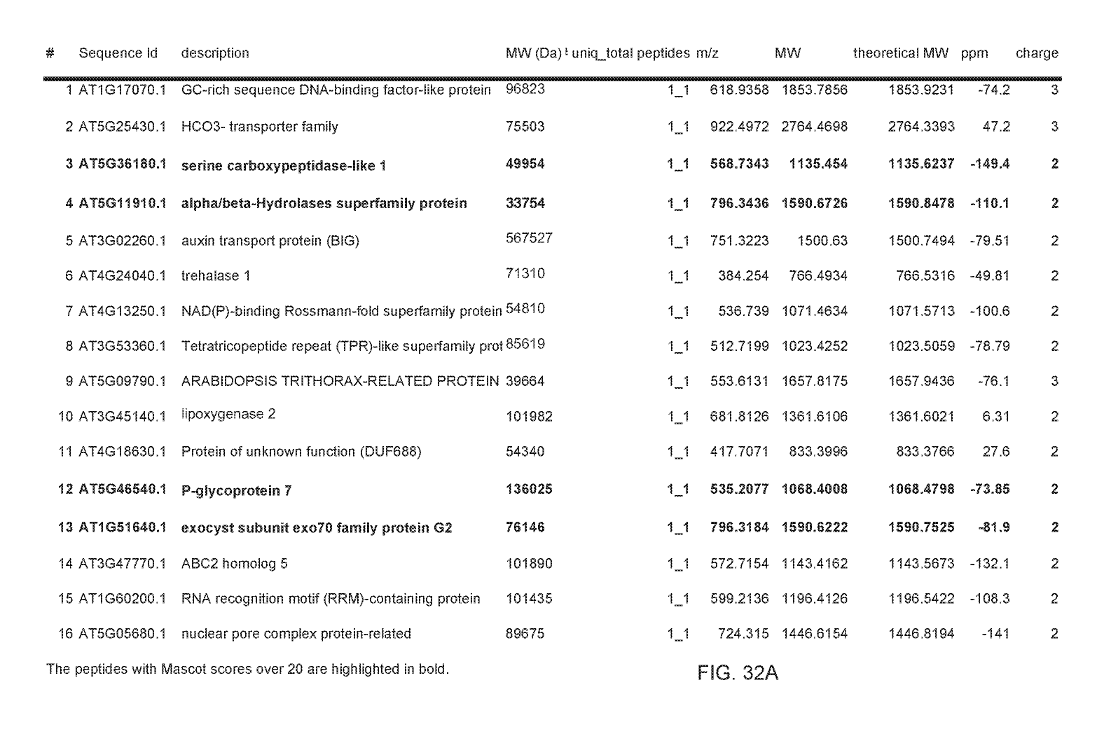

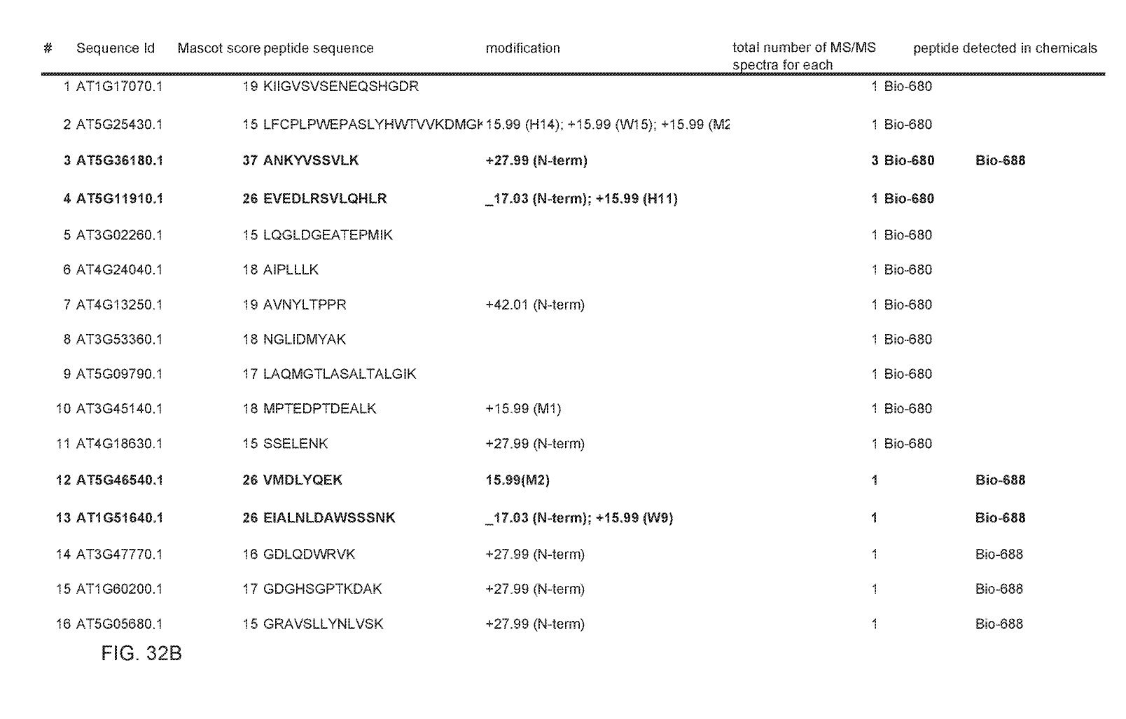

ES2 is a previously identified plant endomembrane trafficking disruptor (FIG. 1A) that inhibits polarized growth of pollen tubes in a dose dependent manner (see FIG. 7A, 7B)¹⁶. Arabidopsis seedlings grown on media containing ES2 have shorter roots, fewer and shorter root hairs and are less sensitive to gravity stimulation (FIG. 7C-7G). ES2 disrupted the trafficking of proteins that are actively recycled between the plasma membrane and endosomes, such as the brassinosteroid receptor (BRI1), the auxin transporters PINFORMED1 (PIN1) and PIN2 after short time treatment (2 hours) (FIG. 8)¹⁶⁻¹⁷. Although ES2 was originally identified from the same phenotype cluster as bioactive compounds ES1 and ES3, it did not target the same proteins as ES1 and ES3 because it did not induce aggregation of Trans-Golgi Network marker SYP61 compared to ES1 and did not affect ROP6 localization compared to ES3, respectively (FIG. 8)^{17,18}. ES2 also did not affect the localization of cellular markers such as HDEL:GFP (Endoplasmic Reticulum, ER), GOT1p:YFP (Golgi), SYP21:YFP (prevacuolar compartment, PVC), PGP4:GFP (plasma membrane) or PIP2a:GFP (plasma membrane) (FIG. 9).

ES2 effects were further explored at the cellular level using GFP-tagged PIN2 protein because it is known to traffic to the plasma membrane, endosomes and vacuoles¹⁹⁻²² Short term ES2 treatment reduced the amount of the plasma membrane localized PIN2 compared to control seedlings (FIG. 1A-E). When the inventors performed ES2 treatment of PIN2::PIN2:GFP expressing seedlings in the dark to inhibit vacuolar-localized GFP fusion protein degradation²³, an increased amount of GFP fluorescence was found in the vacuoles compared to the control (FIG. 1F). The results indicated that ES2 treatment inhibited trafficking to the plasma membrane and enhanced trafficking to the vacuole for degradation.

The feret diameter of PIN2-localized compartments observed from fluorescence confocal microscope images upon ES2 treatment under light conditions was 1.18±0.47 μm (mean±SD, N=391, from 107 cells of 11 seedlings), with a maximum feret diameter of 2.9 μm and a minimal feret diameter of 0.4 μm (FIG. 1G). Size distribution of ES2-induced PIN2 agglomerations indicate that they are very different from known brefeldin A (BFA) induced agglomerations resulting from abnormal trafficking at the Golgi. PIN2 vacuolar trafficking involves the retromer complex, including its component Sorting Nexini that co-localizes with ARA7/RabF2b endosomes^{20,21}. In seedlings expressing both PIN2::PIN2:GFP and ARA7/RabF2b:mRFP, the ES2 treatment was found to induce PIN2 accumulation in the ARA7/RabF2b endosomal compartments (FIG. 1H). 338 PIN2 agglomerations from roots of 12 seedlings treated with ES2 were manually examined and it was found that all of these agglomerations had partial or complete co-localization with ARA7/RabF2b labeled late endosomes/PVC. Accumulation of PIN2 in ARA7/RabF2b-positive compartments was consistent with the inventors' observation that ES2 reduced PIN2 plasma membrane localization and enhanced PIN2 trafficking to the vacuole.

Next examined was whether reduced quantity of PIN2 at the plasma membrane was a result of reduced PIN2 recycling through the GNOM-dependent pathway. The PIN2::PIN2:GFP seedlings grown on normal media were treated with BFA for 2 hours and then permitted to recover in liquid media containing either ES2 or DMSO for another 1.5 hours before imaging. It was found that the disappearance of BFA bodies in ES2 treated seedlings showed a significant delay in comparison to the DMSO containing media (FIG. 1I-J), indicating that ES2 treatment reduced PIN2 recycling through GNOM endosomes. Similar delayed PIN2 recycling has been observed in mutants that are defective in exocytosis²⁴.

The ES2 molecule contains an N-acyl hydrazone group at its core, and could have the propensity for hydrolysis to 3-fluorobenzohydrazide and 4-hydroxy-3-iodo-5-methoxybenzaldehyde in aqueous solution (FIG. 10A). In order to confirm whether ES2 is stable in the inventors system, the stability of ES2 was tested in water solution using ¹H NMR analysis. ¹H NMR spectra of ES2 were collected at different time points over a course of one week, and no hydrolysis products were observed under these conditions (FIG. 10B). In addition, the activity of the two possible ES2 hydrolysis products in inducing PIN2 localization in late endosomal compartments was tested. It was found that PIN2 localization at the plasma membrane after treating PIN2::PIN2:GFP seedlings with 40 μM of the 3-fluorobenzohydrazide or 4-hydroxy-3-iodo-5-methoxybenzaldehyde for two hours, was similar to that observed in the DMSO control, but significantly different to the intracellular localization pattern in ES2 treated samples (FIG. 10C-10F). These data showed that ES2 is a stable compound under aqueous solution and the induced trafficking phenotypes are not due to any in situ hydrolysis byproducts.

Overall, the inventors concluded that ES2 reduced trafficking to the plasma membrane through inhibition of recycling and enhanced protein trafficking to the vacuole. This suggests a possible mechanism to regulate the dynamics of vesicle trafficking.

### EXO70A1 is a Cellular Target of ES2

Structure-activity relationship (SAR) analysis was performed to identify moieties in ES2 that were dispensable for its activity based on the induction of PIN2 localization in agglomerations (FIG. 2A, FIG. 11). It was found that the iodine in the molecule was necessary for its activity while the benzoic ring with the fluorine could accommodate different atoms while maintaining activity. In order to generate analogs with biotin to facilitate target identification, new active and inactive analogs were synthesized with an amine group in the benzoic ring with the fluorine named analog-688 (Ana-688) and analog-680 (Ana-680), as active and inactive analogs respectively (FIG. 2B-C). These two analogs were further modified to produce biotinylated molecules using the amine group and named Bio-688 and Bio-680 respectively (FIG. 2D-E) (see Extended Data method for scheme and FIGS. 18-31 for characterization of synthesized compounds). Ana-688 and Bio-688 induced PIN2 agglomerations after short-term treatment while Ana-680 and Bio-680 did not, indicating they could be used as active analogs and inactive analogs respectively.

Bio-688 and Bio-680 were coupled to streptavidin agarose resulting in active and inactive matrices, respectively, which were incubated with Arabidopsis cell extracts. Proteins bound to the active and inactive matrices were eluted by ES2, and the eluted fractions were analyzed using Mass Spectrometry. Although the peptide abundance in the elution fractions was low (SI. 2), a peptide was detected from Arabidopsis EXO70G2, which belongs to the EXO70 family in Arabidopsis that is involved in exocytosis, from the active matrix but not the inactive matrix elution. EXO70G2 belongs to the EXO70 family that has 23 members in Arabidopsis divided into subclasses A to H^{7,25,26}. EXO70G2 was chosen as a putative candidate target of ES2 because it functions in the same pathway as that is affected by ES2. Other approaches were then taken to test for possible interaction between ES2 and EXO70 proteins in Arabidopsis. Using an available EXO70 antibody, the presence of a close paralog EXO70A1 was tested on the matrix by western blot (FIG. 2F)^{7-9,24,27}. Quantification of the intensity of the Western blot bands indicated that Bio-688 matrix was more potent in pulling down EXO70A1 in comparison to Bio-680 matrix and biotin controls (FIG. 2G) indicating that EXO70A1 interacted more strongly with the active ES2 analog compared to the inactive analog.

A relatively new approach was taken for chemical target identification called Drug Affinity Responsive Target Stability (DARTS) to test the interaction between ES2 and EXO70A1²⁸. The DARTS approach was developed based on the observation that some proteins are protected from degradation by proteases when bound to the ligand²⁸. Arabidopsis protein extract was incubated with ES2 or DMSO and then digested with different concentrations of proteases. After normalizing EXO70A1 protein western blot band intensity against that of the actin internal control, it was found that the degradation of EXO70A1 was significantly protected by ES2 compared to actin which was detected on the same blotting membrane at protease dilutions of 1:3000 and 1:10,000 (FIG. 2H). EXO70A1 protein from E. coli was expressed and purified, then tested for its interaction with ES2 using Saturation-Transfer Difference Nuclear Magnetic Resonance (STD-NMR)²⁹. EXO70A1 amino acid residues 75-638 were used for STD-NMR due to instability of the full-length protein (FIG. 2I). The ¹H assignments of ES2 in DMSO-d₆ were determined from gCOSY and gNOESY spectral analysis (FIG. 12A-B, Table 1). Assignments in D₂O were made by comparison with the 1D spectrum recorded in DMSO-d₆. To determine the optimum saturation time a STD build-up curve was generated using 400 μM ES2 and 20 μM EXO70A1 sample (FIG. 12C). The build-up curve (FIG. 2J) clearly indicated that there was direct interaction between ES2 and EXO70A1. The ¹H spectral from an unrelated molecule showed no interaction with EXO70A1 (FIG. 2J, arrow). Based on the results from the build-up curve all further STD-NMR measurements were taken using a 2 second saturation time. EXO70A1 was titrated with different concentrations of ES2 in STD-NMR experiment and the STD amplification factor was calculated as a function of ES2 concentration (FIG. 2K). The dissociation constant (K_{d}) of EXO70A1 and ES2 interaction using STD-NMR was 400±170 μM and the Bmax was 12.9±2.74 (Table. 2). Also performed were the ¹H assignments of ES2 analog8 (Table 4; FIG. 12D), an inactive analog, and the interaction between analog8 and EXO70A1 was studied using STD-NMR. The calculated STD amplification factors did not show a significant increase with elevated concentrations of analog8 (FIG. 2K). This confirmed the detected interaction between ES2 and EXO70A1 using STD-NMR is not due to random interaction of any small molecule with EXO70A1 protein.

TABLE 1
1H NMR chemical shifts of ES2.

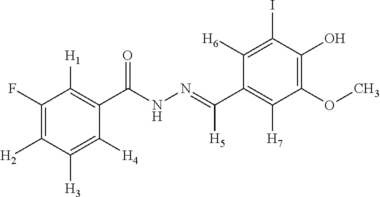

ES2 ¹H Chemical Shifts d(ppm)

| Number | DMSO-d₆ | D₂O |
|---|---|---|
| 1 | 7.70 | 7.49 |
| 2 | 7.45 | 7.29 |
| 3 | 7.59 | 7.46 |
| 4 | 7.76 | 7.55 |
| 5 | 8.29 | 8.06 |
| 6 | 7.61 | 7.59 |
| 7 | 7.33 | 7.33 |
| NH | 10.09 | NA |
| CH3 | 3.89 | 3.79 |
| OH | NA | NA |

TABLE 4
ES2 ana1og8 ¹H NMR chemical shifts.

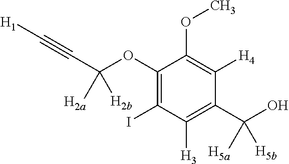

ES2 ¹H Chemical Shifts d(ppm)

| Number | DMSO-d₆ | D₂O |
|---|---|---|
| 1 | 7.70 | 7.49 |
| 2 | 7.45 | 7.29 |
| 3 | 7.59 | 7.46 |
| 4 | 7.76 | 7.55 |
| 5 | 8.29 | 8.06 |
| 6 | 7.61 | 7.59 |
| 7 | 7.33 | 7.33 |
| NH | 10.09 | NA |
| CH3 | 3.89 | 3.79 |
| OH | NA | NA |

In order to further confirm results from STD-NMR, the technique of microscale thermophoresis (MST) was utilized to quantify the dissociation constant for the complex of ES2 (titrant) with EXO70A1 (target molecule). This method observes the motion of molecules in response to a temperature gradient³⁰⁻³². Thermophoresis is characterized by monitoring the time-dependent fluorescence, referred to as a time-trace, of a labeled target molecule in a small zone subject to localized heating by an infrared laser³¹. Multiple time-traces were acquired for serial dilutions of a binding partner. Since binding of the titrant with the target molecule results in a change of mass, charge or hydration entropy, the complex exhibits different thermophoretic behavior than the target molecule alone^{30,32}. Plotting the thermophoretic effect as a function of titrant concentration presents dose-dependent behavior. From the dose-responsive curve, a K_{d} of 253±63.5 μM was calculated for the interaction of ES2 with EXO70A1. While the mean value from MST is lower than the K_{d} from STD-NMR, the results are not significantly different with a 95% level of confidence. This result suggests a micromolar affinity for the binding of ES2 to EXO70A1 consistent between binding assays based on different physical principles (Table 2). Moreover, when MST is performed for the interaction of a negative control, analog8, with EXO70A1, no change was observed in thermophoretic behavior as expected (FIG. 21).

TABLE 2
Non-linear fit summary of ES2 binding
curves using STD-NMR and MST.

| | | MST | |
|---|---|---|---|
| | STD | | EXO70A1L596A; |
| Parameters | EXO70A1 | EXO70A1 | I613A |
| Bₘₐₓ | 12.9 ± 2.74 | 176 ± 26.9 | 96.9 ± 16.9 |
| (μM, mean ± SE) | | | |
| h | 1.21 ± 0.297 | 1.73 ± 0.436 | 4.13 ± 2.85 |
| K_{d} | 400 ± 170 | 253 ± 63.6 | 252 ± 50.6 |
| (μM, mean ± SE) | | | |
| R square (%) | 90.2 | 81.7 | 46.5 |

It was concluded from these different assays that ES2 interacted with EXO70A1 in vitro suggesting that it could be a target in vivo.

### The Expression of EXO70A1 N-Terminus Results in De-Sensitization to ES2

To investigate the relationship between ES2 and the EXO70 gene family at the genetic level, root growth phenotypes of available exo70 mutants as listed in Table 5 were tested in the presence of ES2. None of the 24 mutants that were tested displayed significant differences in response to ES2 when compared with wild type except one. Heterozygous seedlings of T-DNA insertion allele exo70A-3 (SALK_023036) showed resistance to ES2 in root growth (FIG. 3A). In order to study the response of T-DNA insertion mutant plants to ES2 at the cellular level, PIN2::PIN2:GFP were crossed with exo70A-3 heterozygous plants. The F3 population from a F2 seedling that was homozygous for PIN2::PIN2:GFP and heterozygous for T-DNA insertion was used to study the difference between wild type plants and exo70a1-3 heterozygous plants. Upon ES2 treatment, heterozygous T-DNA insertion mutant seedlings showed smaller and fewer PIN2 agglomerations compared with wild type seedlings from the same segregating population (FIG. 3B-E). Although heterozygous exo70A1-3 seedlings had normal growth at the seedling stage the plants had retarded growth after bolting, more shoot stems, reduced seed yield, and abnormal flower development (FIG. 13A). Homozygous exo70a1-3 seedlings had severe growth phenotypes that included arrested root growth and abnormal root meristem organization and were seedling lethal (FIG. 13B), similar to previously characterized exo70A1-1 and exo70A-2 mutant alleles but are more severe⁷. The more severe phenotypes in exo70a1-3 homozygous plants were reported previously, and it was suspected that there was an uncharacterized mutation in this T-DNA insertion line causing the severe phenotypes³³. It was decided to further characterize this allele to find out the linkage between T-DNA insertion in EXO70A1 and ES2 resistance. The homozygous mutant at the transcription level and was found to accumulate the 5′ end of the EXO70A1 mRNA upstream of the T-DNA insertion site whereas the 3′ end of mRNA downstream of the T-DNA insertion was not detected (FIG. 3F-G). It was suspected that this mutation resulted in dominant resistance due to the truncated mRNA encoding a stably accumulated N-terminal peptide that caused the severe phenotypic defect in exo70a1-3. In contrast, exo70A1-1 and -2 were reported as null alleles that exhibit a milder phenotype 7. This led the inventors to characterize the mutant allele at the protein level.

TABLE 5
exo70 mutants tested on ES2

| Gene Name | Gene ID | T-DNA | Insertion location |
|---|---|---|---|
| EXO70A1 | At5g03540 | SALK_014826 | intron |
| | | SALK_135462 | exon |
| | | SALK_086531C | promoter |
| EXO70B1 | At5g58430 | SALK_202386C | exon |
| EXO70B2 | At1g07000 | SALK_129247C | promotor |
| | | SALK_091877C | intron |
| EXO70C1 | At5g13150 | SALK_019833C | 5′UTR |
| EXO70C2 | At5g13990 | SALK_045767C | promotor |
| EXO70D1 | At1g72470 | SALK_049470C | 5′UTR |
| | | SALK_074650 | exon |
| EXO70D2 | At1g54090 | SALK_145760C | promotor |
| EXO70D3 | At3g14090 | SAIL_175_D08 | exon |
| EXO70E2 | At5g61010 | FLAG_36A03 | exon |
| EXO70F1 | At5g50380 | SALK_036927C | exon |
| EXO70G1 | At4g31540 | SALK_090909C | exon |
| EXO70G2 | At1g51640 | SALK_097393C | 3′UTR |
| | | GK_548B11 | exon |
| | | SAIL_292_B03 | exon |
| EXO70H1 | At3g55150 | SALK_042456C | exon |
| EXO70H3 | At3g09530 | SALK_034560 | exon |
| EXO70H4 | At3g09520 | SALK_003200C | exon |
| EXO70H5 | At2g28640 | SALK_007810C | promotor |
| EXO70H7 | At5g59730 | SALK_072673C | exon |
| EXO70H8 | At2g28650 | SALK_109554C | exon |

The expected mass of the truncated peptide was approximately 25 kd with 231 amino acids. Since the anti-EXO70A1 antibodies did not recognize the N-terminal region of EXO70A1, the inventors took advantage of mass spectrometry analysis. The inventors first surveyed E. coli-expressed EXO70A1 protein with nano-LC/MS/MS and identified a peptide ion from its N-terminal region (aa 90-109) with strong signal intensity (FIG. 14). Using this peptide ion with known m/z, charge state and retention time as the fingerprint for EXO70A1, the inventors then analyzed plant proteins derived from wildtype and the T-DNA line. The inventors analyzed SDS gel bands with approximate masses of 70 kd and 25 kd, respectively, from both the wild-type and exo70a1-3 mutant using nano-LC/MS (the system was confirmed with no background after blank injection). It was found that the aa 90-109 fingerprint was detected in wild type 70 kd and exo70a1-3 25 kd samples (FIG. 3H) indicating that the exo70a1-3 allele accumulates a truncated peptide in planta that might be responsible for the ES2 resistance. In order to test the contribution of the truncated peptide to the growth resistance of the exo70A1-3 mutant, the inventors expressed EXO70A1 aa 1-231 in wild type plants under Cauliflower Mosaic Virus 35S promoter then analyzed the effect of ES2 on the transgenic lines. Homozygous transgenic lines expressing the N-terminus of EXO70A1 showed partial resistance to ES2 in root growth when grown on media containing lower concentrations of ES2 (FIG. 3I). The inventors also found resistance to ES2 when EXO70A1-3 heterozygous seedlings or EXO70A1 N-terminus expression seedlings were transferred from normal media to ES2-containing media (FIG. 3J). Reproduction of ES2 resistance in transgenic lines confirmed the linkage between EXO70A1 N-terminus expression and ES2 resistance. Similar to exo70A1-3 heterozygous plants, plants expressing the N-terminal truncated peptide displayed normal growth at the seedling stage in the absence of ES2 but retarded growth, small stature, reduced seed yield, and flower development defects after bolting on soil (FIG. 15). Thus, it was concluded that the EXO70A1 N-terminal peptide was sufficient to induce dominant ES2 resistance in plants, and this confirmed in planta that EXO70A1 was a target of ES2. This suggested that the exocyst could be an important site for controlling the dynamics between recycling to the plasma membrane and vacuole targeting and that the N-terminal domain probably served distinct roles in exocyst regulation. Heterozygous EXO70A1-3 plants show developmental phenotypes in later stages of plant development, which is similar to the EXO70A1 N-terminal peptide expression line. This may also reflect regulatory roles of the EXO70A1 N-terminal domain and the contribution of EXO70A1 to different stages of plant development.

### ES2 Inhibits Cellular Dynamics of EXO70A1

To discover whether ES2 inhibited EXO70 cellular dynamics directly, we examined the cellular localization of GFP-tagged EXO70A1 (GFP:EXO70A1) in Arabidopsis root cells upon ES2 treatment. GFP:EXO70A1 showed plasma membrane localization with distinct polarized maximum at the outer lateral side of root epidermal cells in the root tip (FIG. 4A, white arrows). We found that upon ES2 treatment, the polarized localization pattern of EXO70A1 was lost (FIG. 4A-C). Plot of fluorescence intensity across the root transition zone in DMSO treated control seedlings revealed high fluorescence intensity in the outside layer of the root epidermal cells (FIG. 4B). After two hours of 40 μM ES2 treatment, the lateral polarity of EXO70A1 was changed, as reflected by reduced fluorescence intensity at the outside layer of root epidermal cells (FIG. 4C). However, the lateral polarity pattern of two other exocyst components EXO84 and SEC8 was not affected (FIG. 16A, white arrows). In root hair cells, where active exocytosis is required for polarized growth^{34,31}, it was found that upon ES2 treatment, the fluorescence recovery of GFP:EXO70A1 after photobleaching was significantly slower compared with control cells (FIG. 4D). The maximum fluorescence recovery was more than 80% of the pre-bleached intensity within 3 minutes in control cells, whereas the fluorescence recovery was limited to 20% of the pre-bleached level at the same time in cells treated with ES2 (FIG. 4E). This indicated that ES2 significantly inhibited the cellular dynamics of EXO70A1 in root hair cells as well. The fact that ES2 strongly interfered with EXO70A1 localization without perturbing two other exocyt subunits further supported that in planta EXO70A1 was a target of ES2. The specificity of mis-localization suggests that the mechanism of EXO70A1 lateral plasma membrane targeting is distinct from that of other exocyst components. Furthermore, EXO70A1 may dissociate from the complex independently from other subunits indicating that the maintenance of its lateral polarity may be distinct (assuming that EXO84, SEC8 and EXO70A1 are part of the exocyt while at the lateral plasma membrane). Furthermore only lateral polarity was affected (not isolateral localization) indicating that EXO70A1 lateral polarity is distinct.

### ES2 Targets EXO70 to Inhibit Recycling in Mammalian Cells

Due to the evolutionary conservation of the composition and function of the exocyst complex, the inventors were interested in investigating whether ES2 can target EXO70 in other systems. The inventors examined whether ES2 would affect exocytosis in human cells using the transferrin recycling assay which measures the recycling of endocytosed transferrin to the plasma membrane. The assay has been commonly used to study protein trafficking to the plasma membrane. After treatment with DMSO as a control or ES2 for 1 hour, the cells were pulsed with transferrin (Tfn)-AlexaFluor488 on ice for 5 min and chased with complete media to track Tfn trafficking over time. Most of the Tfn was exocytosed after a 90 min chase in cells treated with DMSO (FIG. 5A). However, Tfn accumulated markedly at the protrusion sites of cells treated with ES2 indicating that exocytosis was reduced (FIG. 5B). In order to test whether the reduced exocytosis in mammalian cells was related to EXO70 as in plants, the inventors tested the localization of GFP-tagged rat EXO70 (rEXO70) protein after the ES2 treatment. It was found that ES2 treatment induced the accumulation of EXO70 vesicles near the plasma membrane (FIG. 5C). A similar effect was observed for human EXO70 isoform2 and isoform5 proteins upon ES2 treatment (FIG. 16B). These vesicles may suggest a block of vesicle tethering and fusion with plasma membrane. To test direct interaction between ES2 and rEXO70, the inventors purified E. coli expressed rEXO70 (FIG. 5D) and performed a STD-NMR experiment. The interaction between ES2 and rEXO70 was confirmed by the presence of saturation energy transfer (FIG. 5E). These data confirm that ES2 directly targets EXO70 in vivo to inhibit exocytosis in mammalian cells as well as in plants. It further indicates that in human cells, ES2 can target multiple isoforms of EXO70 resulting in mis-regulation of exocytosis.

The fact that plant and mammalian EXO70 proteins are targets of ES2 suggests structural similarity. To better understand the structural basis for the conservation of the altered regulation of EXO70A1 by ES2, the inventors crystallized Arabidopsis EXO70A1 and determined its structure at 3.1 Å resolution. The crystal structure of EXO70A1 revealed that it adopted an elongated architecture resembling that previously observed for yeast^{36,37} and mouse EXO70 (mEXO70)³8. The inventors were able to trace 17 α-helices in the structure which were further divided into three domains based on inter-domain hinge points and the overall arrangement of helices: N-terminal (75-379), C-terminal (511-629) and middle (380-510) connecting the N- and C-terminal domains (FIG. 6A)³⁷. The relative conformations of the three domains in EXO70A1 and mEXO70 were similar, except for a slight difference in the orientation of the N-terminal domain. Superposition of the middle-C-terminal domains of EXO70A1 and mEXO70 gave a root mean square deviation (r.s.m.d.) of 1.59 Å on 147 Ca atoms (FIG. 6B). The inventors were able to trace all the helices in the final structure, except that a number of loops connecting the helices are missing due to poor electron density. The statistics for the X-ray diffraction data and structure is summarized in Table 3. Such a high structural similarity suggested that, despite low sequence identity (32% in middle C-terminal domains), the biochemical functions of plant and mammalian EXO70 proteins were most likely conserved. This supported the inventors conclusion that ES2 targets EXO70 in plants and three mammals (rats, humans and probably mice).

TABLE 3
Data collection and refinement statistics

| | EXO70A1(Se-MET) | |
|---|---|---|
| Data collection | | |
| Space group | P2₁2₁2₁ | |
| Cell dimensions | | |
| a, b, c (Å) | 55.1, 72.1, 327.9 | |
| a, b, g (°) | 90, 90, 90 | |
| Wavelength | 0.9774 | |
| Resolution (Å) | 50.0-3.40 | (3.52-3.40) ^{a} |
| R_{sym} or R_{merge} | 0.097 | (0.404) |
| I/sI | 19.7 | (3.3) |
| Completeness (%) | 99.9 | (99.7) |
| Redundancy | 4.6 | (4.3) |
| Refinement | | |
| Resolution (Å) | 48.5-3.40 | |
| No. reflections | 34418 | |
| R_{work}/R_{free} | 0.312/0.337 | |
| No. atoms | | |
| Protein | 5886 | |
| B-factors | | |
| Protein | 116.1 | |
| R.m.s. deviations | | |
| Bond lengths (Å) | 0.010 | |
| Bond angles (°) | 1.497 | |
| Ramachandran plot | | |

| | |
|---|---|
| Favoured regions (%) | 94.0 |
| Allowed regions (%) | 5.8 |
| Outliers (%) | 0.2 |

^{a} Values in parentheses are for highest-resolution shell.

With an available crystal structure of EXO70A1, a molecular docking tool, Autodock, was applied to predict possible ES2 binding sites of the EXO70A1, by fixing the protein and allowing ES2 to freely bind to several potential pockets of the EXO70A1. The inventors decided to use molecular docking to predict possible ES2 binding sites on EXO70A1. Using Autodock³⁹, the inventors found one possible binding pocket located at the C-terminus of EXO70A1. The binding cavity was principally composed of the hydrophobic amino acids Y592, L596, K597, R598, I613 and T616 (FIG. 6c). The conformation of ES2 can fit well in the C-terminal pocket of the wild-type EXO70A1 protein. The fluorine-containing aromatic ring of ES2 locates in the cavity created by P601, R598 and K597 main chains. The iodine-containing aromatic ring is surrounded by hydrophobic sidechains of several residues, including I613, L596, Y592 and T616. When the docking was performed with mutations of L596 and I613 to Ala, the binding pocket became larger because of the missing of I613 and L596 sidechains. However, in this case, the ES2 conformation couldn't fit the pocket properly (FIG. 6D). Although the iodine-containing aromatic ring remained deeply in the pocket and formed interactions with A613, fewer interactions between the fluoride-containing aromatic ring and Y592/A596 were shown. In addition, in contrast to wild-type EXO70A1, the L596A and I613A mutations result in missing attractions between the ES2 and P601, R598 and T616, which would generate higher binding free energy and a weaker ligand-binding mode. In order to confirm that the predicted pocket plays a role in ES2 binding, the inventors mutated amino acids L596 and I613 to Alanine (L596A;I613A) and tested for the binding activity of mutant protein to ES2 using STD-NMR. It was found that under the same protein and ES2 concentrations as used previously, L596A;I613A had less binding to ES2, which was reflected by reduced STD-NMR integral integrity (FIG. 6E-F, FIG. 17A-B). This was consistent with the prediction that L596 and I613 participate in interaction with ES2, although the mutations did not completely abolish the interaction between EXO70A1 and ES2. The inventors also compared the thermophoretic mobility of EXO70A1 and EXO70A1-L596A;I613A when titrated with ES2 (FIG. 6G). Although the maximal thermophoretic mobility was lower for the mutated protein than for EXO70A1, the K_{d} of 252+50.6 for the interaction of ES2 with EXO70A1-L596A;I613A is not significantly different at a 95% level of confidence than the K_{d} for ES2 with EXO70A1 (Table 2). Despite this result, the non-linear fit for the interaction of ES2 with EXO70A1 is more robust with a R-squared of 81.7% than for the interaction of ES2 with EXO70A1-L596A;I613A with a R-squared of 46.5%. This observation suggested that amino acids L596 and I613 were not essential for binding of ES2 but may interact with binding site residues and indirectly affect the local binding microenvironment. Amino acids L596 and I613 are conserved between Arabidopsis EXO70A1 and mammalian EXO70 proteins (FIG. 17C), explaining why ES2 could interact with both Arabidopsis EXO70A1 and rat EXO70.

In summary, the novel small molecule ES2 directly interacts with and inhibits the dynamics of the evolutionary conserved EXO70 proteins to reduce exocytosis in plants and mammals and enhance plant vacuolar trafficking. Expression of the EXO70A1 N-terminus in wild type plants partially overcomes the effects of ES2 indicating that this region might positively regulate plasma membrane docking of the full-length protein. In spite of the high divergence in their primary protein structure, plant and mammalian EXO70 proteins share an evolutionarily conserved structure that likely permits ES2 to target EXO70 proteins in plants and humans. This is the first report of the structure of a plant EXO70 subunit. The similarities in 3-D structure strongly support a conservation of exocyst function and functional sites during evolution. Despite these new details, it is unclear how the exocyt is integrated within the context of the endomembrane trafficking system in multicellular organisms. In Arabidopsis there are 23 genes encoding EXO70 isoforms, more than in mammals or yeast. Most of these isoforms are poorly defined in terms of function. The inventors result in human cells is powerful in that it indicates that ES2 will probably target many EXO70 isoforms and showed the power of chemical genomics in addressing genetic redundancy. It also indicates that by using a high content, cell biology-based pollen screen for modulators of endomembrane trafficking, a molecule was found that will permit the inventors to investigate the basic functional domains of EXO70.

This approach also provides a new avenue toward novel drugs. To date the inventors are not aware of any small molecules that target the exocyst; thus this presents a novel drug target. The detailed role of the exocyst in human diseases requires further investigation. It is contemplated to increase the potency and modify the isoform specificity of ES2 or similar molecules targeting EXO70 to control EXO70-related human diseases such as cancer cell invasions and diabetes, which involves glucose transport. Although the sequence identity between plant EXO70s and yeast EXO70 is lower than that of mammalian cells, the inventors found I613 is conserved between plant and yeast EXO70s. It is contemplated that ES2 can target yeast EXO70 as well and thus can be a tool in fungal pathogen manipulation.

### Example 2

### Materials and Methods:

### Plant Strains and Growth Conditions

Arabidopsis ecotype Col-0 was used as wildtype. The Arabidopsis Col-0 T-DNA insertion mutant SALK_036026 (exo70A1-3) was obtained from the Arabidopsis Biological Resource Center (Columbus, Ohio). PIN2::PIN2:GFP;Ara7:mRFP line is a gift from Dr. Jiri Friml (IST Austria, Vienna).

Half-strength Murashige and Skoog medium (0.5×MS) [0.5×Murashige and Skoog salts, 1% sucrose (pH 5.7)] was used as growth media. For plants that were grown on solid 0.5×MS media, 0.8% phytoagar (Research Products International, IL) was included. To test chemical's growth effect, the chemicals were added to 0.5×MS solid media at 55° C. before pouring the plate. For short-term chemical treatment, the seedlings were grown on 0.5×MS solid media for 5 to 7 days and then transferred to 24-well plates containing 0.5×MS liquid media and chemicals. For dark treatment of PIN2::PIN2:GFP seedlings in DMSO or ES2, the seedlings were grown on 0.5×MS solid media for 5 days and the seedlings were treated with ES2 or DMSO in the dark for 4 hours before imaging with confocal microscopy. All plants, including soil grown plants, were grown in an environmental chamber at long-day lighting conditions (16 hours light/8 hours dark) and a temperature of 22° C. The root hair growth phenotypes were documented using a SPOT camera (SPOT Imaging Solutions, MI) connected to a dissecting microscope.

Total RNA isolation, reverse transcription and polymerase chain reaction analysis of EXO70A transcript in exo70A1-3 followed the published protocol 40. The sequences of primers used in RT-PCR are: EXO70A1 F1: ccATGGCTGTTGATAGCAGA; EXO70A1 R1: CGGAGGAGATCGAATTGAGA; EXO70A1 R2: TGGCTATAGCATCCCCAAAG; EXO70A1 F3: GATGGAACTGTCCACCCACT; EXO70A1 R3: ACCCAATCATCGCCTAACAA; Actin 2 F: GTTTTGCGTTTTAGTCCCATTGT; Actin 2 R: ACAAAAGGAATAAAGAGGCATCAATT.

### In Vitro Pollen Germination and Chemical Treatment

Pollens from soil grown Arabidopsis plants were dusted on a solid medium (18% sucrose, 0.01% boric acid, 1 mM CaCl2), 1 mM Ca(NO3)2, 1 mM MgSO4, pH 6.4 and 0.5% agar) and incubated at 28° C. for 3 hours before observation under an inverted microscope (Nikon eclipse TE300). Images were taken by a cooled CCD camera (Hamamatsu CA4742-95) attached to the microscope. To measure the length of pollen tubes treated by ES2, 8 mM ES2 stock solution were added to the pollen medium to a final concentration of 2 μM, 4 μM, 8 μM or 16 μM before dusting the pollens on the medium. ImageJ software (http://rsb.info.nih.gov/ij) was used for measuring the length of pollen tubes.

### Confocal Microscopy and Image Quantification

Fluorescence imaging was performed using a Leica TCS SP5 confocal microscopy (Leica Microsystems, Wetzlar, Germany). Manufacture default settings were used for imaging GFP-, RFP- and YFP-tagged proteins. To image FM4-64 stained cells, laser line 543 nm was used for excitation and emission light with wavelength 600-700 nm was collected. For BFA washout experiments, seedlings of 5 days old were treated with 40 μM BFA for 2 hours and quickly washed for three times with the normal media. The treated seedlings were transferred to normal media containing 0.5% DMSO or 40 μM ES2 and recovered for 90 minutes before imaging with confocal microscopy.

To quantify the size of PIN2 agglomerations induced by ES2 treatment, 5 days old PIN2::PIN2:GFP seedlings were treated with 40 μM ES2 for 2 hours and the root epidermal cells in the meristem zone were imaged with confocal microscopy. Z-stacks images that cover the entire volume of the epidermal cells were collected. From each Z-stack image, a few adjacent image slices that do not have overlapped agglomerations in XY directions nor Z direction were selected and a maximum Z-projection image was generated by ImageJ software (http://imagej.nih.gov/ij/). The maximum Z-projection images were thresholded to get rid of the diffusive fluorescence and the agglomerations within a single cell were manually selected and then measured by “Analyze Particles” function of ImageJ. The agglomerations with maximum diameter of less than 2 pixels were discarded during statistic analysis.

To measure the colocalization between ES2 induced PIN2 agglomerations with RabF2b/Ara7, 5 days old seedlings of PIN2::PIN2:GFP;RabF2b/Ara7:RFP were treated with 40 μM ES2 for 2 hours and the root epidermal cells in the meristem zone were imaged by confocal microscopy under line sequential scanning mode for GFP and RFP in xyz directions. The collected two-channel Z-stack images were thresholded in both channels to get rid of the background fluorescence and were then analyzed by Colocalization plugin in ImageJ. The resulted Z-stack images were examined manually to find whether each PIN2 agglomeration is associated with a punctate RabF2b/Ara7 structure. A total of 120 cells from 12 individual seedlings were examined.

To analyze the effect of ES2 on EXO70A1 dynamics in root hair cells, FRAP module in SP5 confocal microscope was used. Seven days old GFP:EXO70A1 seedlings were treated with 0.05% DMSO or 4 μM ES2 for 1 hour. Root hairs that have a horizontal orientation and are not twisted by the glass slide and cover slip were selected and the image plane was focused on the region where the root hair width is at maximum. The Region Of Interests (ROI) for photobleaching was selected by freehand selection tool to include the plasma membrane and cytosolic pool of GFP:EXO70A1.

### Gravitropic Response Assays

The gross gravitropic assays were performed as in⁴¹. In brief, wildtype seeds were plated on normal media containing DMSO or ES2, stratified for two days and then light-treated for 8 hours. Plates were then placed vertically in the dark. After three days of growth, plates were rotated 90°. After another two days of growth, seedlings were documented using an Epson scanner (Model 2450, Long Beach, Calif.) and the angles of roots curvature were quantified using ImageJ. The gravitropic root response was also observed using high temporal resolution imaging. Five-day old seedlings of wildtype were re-oriented by rotating the plates 90°. High temporal resolution images captured root curvature every 2 minutes for 8 hours using an AVT Marlin camera (Stadtroda, Germany). Images were exported and root curvature after gravity stimulation was measured by a MATLAB based custom image analysis software⁴². Root curvature was then graphed as a function of time.

### Schemes of ES2 Analogs Biosynthesis

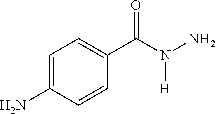 [SMILES: NNC(=O)c1ccc(N)cc1]

### 4-Aminobenzhydrazide

Methyl 4-aminobenzoate (2.0 g, 13.2 mmol) was added to a 10 mL round bottom flask with stir bar, followed by addition of anhydrous hydrazine (2.0 mL, 63.7 mmol). The reaction was then heated to 70° C. under N₂ for 12 hours. After cooling, the mixture was poured into deionized water (100 mL), and the resulting precipitate was filtered and rinsed with additional water (100 mL) to give the product as a white solid (1.42 g, 71%). ¹H NMR (400 MHz; DMSO-d₆) δ=9.26 (s, 1H), 7.54 (d, J=8.6 Hz, 2H), 6.52 (d, J=8.6 Hz, 2H), 5.57 (s, 2H), 4.26 (br s, 2H). ¹³C NMR (100 MHz; DMSO-d₆)=166.4, 151.5, 128.4, 119.9, 112.6. (ESI) m/z calcd for C₇H₁₀N₃O ([M+H]⁺), 152.0818, found 152.0814.

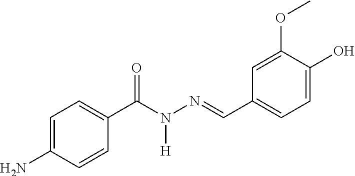 [SMILES: COc1cc(/C=N/NC(=O)c2ccc(N)cc2)ccc1O]

### 4-Amino-N′-[(E)-(4-hydroxy-3-methoxyphenyl)methylidene]benzohydrazide

4-Aminobenzhydrazide (153 mg, 1.01 mmol) and vanillin (154 mg, 1.01 mmol) were added to a 25 mL round bottom flask with stir bar and attached reflux condenser, followed by acetonitrile (10 mL) and AcOH (2 drops). The reaction was then heated to reflux under N₂ for 8 hours. After cooling, a precipitate was filtered, rinsed with iPrOH (50 mL) and dried to give the product as a white solid (205 mg, 71%). ¹H NMR (400 MHz; DMSO-d₆) δ=11.24 (s, 1H), 9.46 (s, 1H), 8.28 (br s, 1H), 7.65 (d, J=8.6 Hz, 2H), 7.27 (s, 1H), 7.04 (dd, J=8.2, 1.8 Hz, 1H), 6.83 (d, J=8.1 Hz, 1H), 6.58 (d, J=8.6 Hz, 2H), 5.74 (br s, 2H), 3.83 (s, 3H). ¹³C NMR (100 MHz; DMSO-d₆)₆=162.9, 152.1, 148.6, 148.0, 146.6, 129.3, 126.2, 121.8, 119.8, 115.4, 112.6, 108.8, 55.5. (ESI) m/z calcd for C₁₅H₁₅N₃NaO₃ ([M+Na]⁺) 308.1006, found 308.1013.

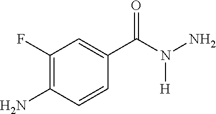 [SMILES: NNC(=O)c1ccc(N)c(F)c1]

### 4-Amino-3-fluorophenylhydrazide

Methyl 4-amino-3-fluorobenzoate (250 mg, 1.48 mmols) was added to a 10 mL round bottom flask with stir bar, followed by addition of anhydrous hydrazine (500 μL, 15.9 mmols). The reaction was then heated to 70° C. under N₂ for 15 hours. After cooling, the mixture was poured into deionized water (40 mL), and the resulting precipitate was filtered and rinsed with additional water (50 mL) to give the product as a tan solid (130 mg, 52%). ¹H NMR (400 MHz; DMSO-d₆) δ=9.41 (s, 1H), 7.48 (dd, J=12.8 Hz, 1.7 Hz, 1H), 7.43 (dd, J=8.6, 1.7 Hz, 1H), 6.74 (t, J=8.6 Hz, 1H), 5.66 (br, 2H), 4.36 (br s, 2H). ¹³C NMR (100 MHz; DMSO-d₆) δ=165.4, 149.5 (d, J=236.8 Hz), 139.5 (d, J=12.9 Hz), 124.0, 120.3 (d, J=4.8 Hz), 114.8 (d, J=3.9 Hz), 113.7 (d, J=19.6 Hz). ¹⁹F NMR (376 MHz; DMSO-d₆) δ=−136.96 (dd, J=12.0, 9.2 Hz). Referenced against CF₃COOH at −76.55 ppm. (ESI) m/z calcd for C₇H₈FN₃O (M⁺) 169.0645, found 169.1001.

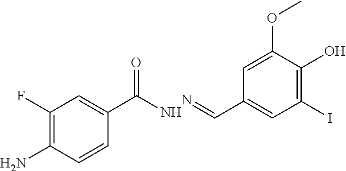

### 4-Amino-3-fluoro-N′-[(E)-(4-hydroxy-3-iodo-5-methoxyphenyl)methylidene]benzohydrazide

4-Amino-3-fluorophenylhydrazide (115 mg, 0.68 mmol) and 5-iodovanillin (189 mg, 0.68 mmol) were added to a 25 mL round bottom flask with stir bar and attached reflux condenser, followed by acetonitrile (10 mL) and AcOH (2 drops). The reaction was then heated at 82° C. under N₂ for 8 hours. After cooling, a precipitate was filtered, rinsed with iPrOH (50 mL) and dried to give the product as a white solid (248 mg, 85%). ¹H NMR (400 MHz; DMSO-d₆) δ=11.48 (s, 1H), 10.00 (s, 1H), 8.24 (br, 1H), 7.56 (m, 3H), 7.30 (s, 1H), 6.80 (t, J=8.7 Hz, 1H), 5.85 (s, 2H), 3.87 (s, 3H). ¹³C NMR (100 MHz; DMSO-d₆)=161.9, 150.5, 148.1, 145.4, 140.2 (d, J=13.1 Hz), 129.85, 127.9, 124.9, 119.9, 114.8, 114.3 (d, J=20.4 Hz), 109.0, 84.5, 56.1. ¹⁹F NMR (376 MHz; DMSO-d₆) δ=−121.36 (m). Referenced against p-difluorobenzene at −106.0 ppm. (ESI) m/z calcd for C₁₅H₁₂FIN₃O₃ (M⁺) 427.9902, found 427.9922.

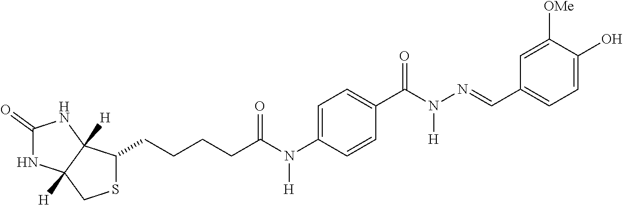

### N-(4-((E)-2-(4-hydroxy-3-methoxybenzylidene)hydrazinecarbonyl)phenyl)-5-((3aS,4S,6aR)-2-oxohexahydro-1H-thieno[3,4-d]imidazol-4-yl)pentanamide

Biotin (51 mg, 0.21 mmol) and HCTU (85 mg, 0.21 mmol) were combined in a 25 mL round bottom flask with stir bar and attached reflux condenser, followed by acetonitrile (18 mL), acetone (3 mL), and Et₃N (200 μL). This was purged with N₂ and the reaction stirred at room temperature for 2 h. 4-Amino-N-[(E)-(4-hydroxy-3-methoxyphenyl)methylidene]benzohydrazide (60 mg, 0.21 mmol) was then added, followed by heating the reaction to 50° C. for 20 h. After cooling to −25° C. a precipitate was centrifuged, followed by filtration and washing with EtOAc (20 mL) to give product as a light yellow solid (24 mg, 22%). ¹H NMR (400 MHz; DMSO-d₆) S=11.47 (br s, 1H), 8.38 (br s, 2H), 8.06 (s, 1H), 7.67 (d, J=8.4 Hz, 2H), 7.42 (s, 1H), 7.24 (d, J=8.0 Hz, 1H), 7.15 (d, J=8.1 Hz, 1H), 6.59 (d, J=8.3 Hz, 2H), 6.45 (br s, 1H), 6.36 (br s, 1H), 5.78 (s, 1H), 4.65 (s, 1H), 4.31 (t, J=5.3 Hz, 1H), 4.13 (m, 1H), 3.84 (s, 3H), 2.85 (dd, J=12.5, 4.9 Hz, 1H), 2.63-2.56 (m, 3H), 1.74-1.40 (m, 6H). ¹³C NMR (100 MHz; DMSO-d₆) 171.0, 162.7, 152.3, 151.2, 140.5, 133.6, 129.6, 129.3, 128.6, 123.2, 120.2, 119.4, 112.6, 109.6, 61.1, 59.2, 55.9, 55.4, 33.0, 28.0, 27.8, 24.5. (ESI) m/z calcd for C₂₅H₂₉NaN₅O₅S ([M+Na]⁺) 534.1782, found 534.1776.

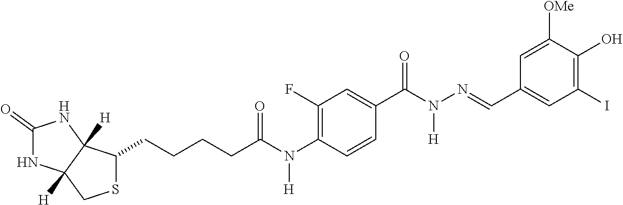

### N-(2-fluoro-4-((E)-2-(4-hydroxy-3-iodo-5-methoxybenzylidene)hydrazinecarbonyl)phenyl)-5-((3aS,4S,6aR)-2-oxohexahydro-1H-thieno[3,4-d]imidazol-4-yl)pentanamide

Biotin (51 mg, 0.21 mmol) and HCTU (85 mg, 0.21 mmol) were combined in a 10 mL round bottom flask with stir bar and attached reflux condenser, followed by acetonitrile (8 mL), acetone (1.5 mL), and Et₃N (200 μL). This was purged with N₂ and the reaction stirred at room temperature for 2 h. 4-Amino-3-fluoro-N-[(E)-(4-hydroxy-3-iodo-5-methoxyphenyl)methylidene] benzohydrazide (88 mg, 0.21 mmol) was then added, followed by heating the reaction to 50° C. for 15 h. After cooling a precipitate was filtered, followed by rinsing with additional MeCN (15 mL) and drying to give product as an off-white solid (83 mg, 60%). ¹H NMR (400 MHz; DMSO-d₆) S=11.98 (br s, 2H), 11.46 (br s, 1H), 9.99 (s, 1H), 8.24 (br s, 1H), 7.56 (ddd, J=25.2, 8.4, 1.7 Hz, 1H), 7.56 (d, J=1.4 Hz, 1H), 7.29 (d, J=1.4 Hz, 1H), 6.80 (t, J=8.7 Hz, 1H), 6.42 (s, 1H), 6.35 (s, 1H), 5.84 (br s, 1H), 4.30 (dd, J=7.7, 4.8 Hz, 1H), 4.13 (ddd, J=7.6, 4.5, 1.4 Hz, 1H), 3.10 (ddd, J=10.5, 6.0, 2.5 Hz, 1H), 2.82 (dd, J=12.4, 5.1 Hz, 1H), 2.58 (d, J=12.4 Hz, 1H), 2.20 (t, J=7.3 Hz, 2H), 1.66-1.27 (m, 5H). ¹³C NMR (100 MHz; DMSO-d₆) δ=174.4, 162.7, 161.8, 150.5, 148.0, 147.3, 145.4, 144.6, 140.2 (d, J=13.1 Hz), 129.8, 127.9, 124.9, 119.9 (d, J=4.7 Hz), 114.7, 114.3 (d, J=19.4 Hz), 109.0, 84.5, 61.0, 59.2, 56.1, 55.4, 39.9, 33.5, 28.1, 28.0, 24.5. ¹⁹F NMR (376 MHz; DMSO-d₆) δ=−121.37 (m). Referenced against p-difluorobenzene at −106.0 ppm. (ESI) m/z calcd for C₂₅H₂₅FIN₅O₅S ([M+H]⁺) 656.0834, found 656.0832.

### STD-NMR Experiments

NMR spectra were collected at 25° C. using a Bruker Avance spectrometer operating at 600 MHz proton frequency with a TXI 5 mm probehead with a z-gradient. The standard Bruker pulse program stddiffesgp.3 was used for data collection employing a 2 second STD saturation time. Spectral acquisition and processing parameters are similar to those used in the reference⁴³. A sample prepared in 500 μl buffered D₂O, containing 20 μM EXO70A1 protein and 400 μM ES2 was used for the initial STD-NMR experiment. To prevent precipitation of ES2 in the D₂O solutions a 5 mM ES2 stock solution was made with DMSO-d₆ as solvent and added to the protein solution. The D₂O buffer solution contained 50 mM Tris.HCl (PH 8.0) and 150 mM NaCl.

### MST Experiment

MST experiments were carried out using a Monolith NT.115 (NanoTemper Technologies GmbH, Munich, Germany). Purified E. coli expressed EXO70A1 and EXO70A1-L596A;I613A were fluorescently labeled with NT-647 (available from NanoTemper Technologies GmbH) via amine conjugation. Increasing concentrations of titrant (either ES2 or ES2 analog 8) were titrated against constant concentrations (50 nM) of labeled, target protein (either EXO701A or EXO701A-L596A;I613A) in a standard MST buffer (50 mM Tris pH7.5, 150 mM NaCl, 10 mM MgCl2, 0.05% Tween-20). The small molecules were dissolved in DMSO for a final concentration of 7.5% when added to an equal volume solution of target protein. MST premium-coated capillaries (Monolith NT.115 MO-K005) were used to load the samples into the MST instrument. Triplicate time-traces were acquired for each test system. For each titration, two controls were measured to observe the thermophoretic response of the target protein when titrant was not present. The mean thermophoretic response of the controls was subtracted from the thermophoretic response of the target protein in the presence of the titrant. The resulting data was processed with the Graphpad Prism software (Graphpad, La Jolla, Calif.). Data was fit to the Hill equation using least-squares, non-linear regression to calculate the max binding (Bmax), Hill coefficient (h), and dissociation constant (K_{d}). K_{d} values were further compared between all test systems using one-way ANOVA within Graphpad Prism.

### Pull Down Assay Using Biotin Tagged Molecules and Mass Spectrometry Analysis of Bound Proteins

In order to pull down proteins bound to Biotinylated ES2 analogs, protein extracts from 16 days old seedlings grown from normal 0.5×MS agar media with the plates in vertical orientation were used. 4 ml extraction buffer (1×PBS, 0.5% Triton x-100, 2 mM DTT, 1×protease inhibitor cocktail) was added to the tissue powder (ground in liquid nitrogen) resulted from 2 grams of seedlings. The cell extracts were passed through 2 layers of miracloth and the flow through was first spun at 1000×g for 30 minutes. The supernatant from 1000×g was collected and spun at 16,000×g for 15 minutes. The resulted 16,000×g supernatant fraction was used as protein input during pull down. High Capacity Streptavidin agarose resins (Thermo Scientific, Rockford, Ill.) were equilibrated with protein extraction buffer and then incubated with 100 μM biotin-tagged ES2 analogs at room temperature with gentle end-to-end inverting for 1 hour. The Streptavidin resins were collected and incubated with 2 ml protein extract for 2 hours at room temperature with end-to-end inverting. The Streptavidin resins were then washed with extraction buffer for 3 times and the putative ES2 binding proteins were eluted with 100 μl extraction buffer contains 100 μM ES2 by end-to-end inverting for 1 hour at room temperature. The eluted proteins were digested with 1 μg of trypsin. Tryptic peptides were analyzed with a 5-fraction MudPIT method described in a previous study⁴⁵. To detect EXO70A1 protein on the Streptavidin resins after pull down, 1×SDS loading buffer were added to the resins and boiled for 5 minutes. The entire resins fractions were loaded to SDS-PAGE for western blotting using anti-EXO70A1 antibody.

Mass Spectrometry Detection of EXO70A1 N-Terminal Peptide Fingerprints in Wildtype and exo70a1-3

To verify that the mRNA corresponding to the N-terminus of EXO70A1 is truly translated into a polypeptide in exo70a1-3 cells, we isolated total proteins from 10 days old wildtype and exo70a1-3 homozygous seedlings using the same procedure as used in pull down assay. The total proteins were separated on SDS-PAGE and then stained with coomassie blue. Proteins with the molecular weight of around 70 kilodalton (kd) (corresponding to full-length EXO70A1) and 25 kd (corresponding to N-terminal portion of EXO70A1) were excised from the stained gel using a razor blade. The gel bands were processed and treated with trypsin as described⁴⁶. Because EXO70A1 is relatively low abundant in cells, a general proteomics profiling of the gel bands is not a suitable method to be able to determine the presence of EXO70A1 in the samples. With a targeted analysis method⁴⁷, an E. coli-expressed EXO70A1 was used to determine that a peptide ion corresponding to a.a. 90-109 showed strongest signal intensity in an nanoLC/MS spectrum. After long hours of extensive washing until there was not any detectable signals due to column carry-over, then samples from gel bands were injected and analyzed with nanoLC/MS for the detection of this signature peptide ion, which served as an evidence of the presence of either full-length EXO70A1 or its N-terminus. Subsequently, further nanoLC/MS/MS was also performed only for this ion to confirm its amino acid sequence.

### DARTS Assay

We followed the published protocol for DARTS assay⁴⁸. Protein extract used for DARTS assay was obtained using the same protocol as the pull down assay. In brief, 300 μl protein extracts were incubated with either 400 μM ES2 or 1% DMSO for 1 hour at room temperature. This was divided into 6 aliquots of 50 μl to which different concentrations of pronase (Sigma, St. Louis, Mo.) were added, and digested for 30 minutes at room temperature. We then added SDS-loading buffer and boiled the samples to stop the reaction. The denatured samples were loaded to SDS-PAGE and the same membrane was probed with anti-EXO70A1 and anti-actin antibodies. The resulted x-ray films were scanned and quantified using Image J. The signal intensity of each lane was calculated and subtracted from background signal and the ratio between ES2 treated sample and DMSO treated sample at each of the pronase concentration was calculated.

### Expression, Purification and Crystallization of EXO70A1

The cDNA sequence encoding EXO70A1 (residues 75-638) was amplified by PCR and sub-cloned into a modified pRSFDue6-1 vector, in which it was separated from a preceding hexa-histidine-SUMO tag by a ULP (Ubiquitin Like Protease 1) cleavage site. The plasmid containing the fusion protein was transformed into BL21(DE3) RIL cell strain (Novagen Inc) for overexpression. The cells were grown at 37° C. and induced by 0.05 mM isopropyl β-D-1-thiogalactopyranoside (IPTG) when the OD₆₀₀ reached 0.6. After induction, the cells continued to grow at 20° C. overnight. The fusion protein was purified using a Ni-NTA column. Subsequently, the His6-SUMO tag was removed by ULP cleavage, followed by a second Ni-NTA run. The EXO70A1 protein was finally purified through size exclusion chromatography on a Superdex 200 16/60 column. The protein sample was concentrated to ˜17 mg/ml and stored in a buffer containing 50 mM Tris-HCl, pH8.0, 300 mM NaCl and 5% glycerol. For crystallization, Selenium Methionine (SeMet)-labeled EXO70A1 was expressed in M9 minimum medium and purified in the same way as described above.

SeMet-labeled EXO70A1 was crystallized using sitting-drop vapor diffusion method by mixing 1.5 μL protein and 1.5 μL reservoir solution containing 200 mM di-ammonium tartrate, pH7.0, and 21.5% PEG3350 at 16° C. Crystals that grew into full size in a week were equilibrated in reservoir solution supplemented with 25% glycerol before quick-frozen in liquid nitrogen. The X-ray diffraction data was collected at beamline 5.0.1. at the Lawrence Berkeley National Lab Center for Structural Biology (BCSB), and integrated and scaled by HKL2000 package. The structure was solved by the SAD method, with 23 out of the 26 Selenium atoms found in the two protein molecules per asymmetric unit. The initial model of EXO70A1 was built in Coot⁴⁹, followed by iterative cycles of model rebuilding and refinement using COOT and PHENIX⁵⁰. TLS refinement was applied to improve the electron density map.

### Computational Details of Docking Simulations

To better understand ligand-bound conformation and the effect of I613A and L596A mutation in an EXO70-ES2 system, we employed an Autodock program³⁹ with Lamarckian genetic algorithm to execute ligand docking by fixing a protein and allowing an ES2 to move around I613 and L596 at the C-terminal of EXO70A1. The 3-dimensional (3D) experimental coordinate of EXO70A1 crystal structure was obtained in this study. We created a 3D structure of ES2 by using the VegaZZ program⁵¹. The Autodock scoring function is a subset of the AMBER force field that treats molecules using the United Atom model; and Gasteiger charges⁵² was assigned to the molecules by applying Autodock tools 1.5.4⁵³. The ES2 docking simulations were performed on the two types of EXO70A1: One is the protein with wild-type sequence and the other protein containing I613A and L596A mutations. Autogrid version 4.0 was used to create affinity grids with 0.375 Å spacing. The cubic grid box with a dimension of 2.25 nm was centered near the EXO70A1 C-terminus. In each molecular docking, we trailed 10 docking simulations; and one million energy evaluations for each trail were performed.

### Example 3

FIGS. 18-31 provide ¹H, ¹³C and ¹⁹F NMR spectra of the synthesized compounds. In FIG. 32, Table 6 provides information on the peptides detected from the pull-down assay using biotin-tagged analogs combined with Mass Spectrometry analysis.

### REFERENCES

The following publications are incorporated by reference herein:
1. Novick, P., Field, C. & Schekman, R. Identification of 23 complementation groups required for post-translational events in the yeast secretory pathway. Cell 21, 205-15 (1980).
2. Zhao, Y. et al. Exo70 generates membrane curvature for morphogenesis and cell migration. Dev Cell 26, 266-78 (2013).
3. Fujita, A. et al. GTP hydrolysis of TC10 promotes neurite outgrowth through exocytic fusion of Rab 11- and L1-containing vesicles by releasing exocyst component Exo70. PLoS One 8, e79689 (2013).
4. Dupraz, S. et al. The TC10-Exo70 complex is essential for membrane expansion and axonal specification in developing neurons. J Neurosci 29, 13292-301 (2009).
5. Zuo, X. et al. Exo70 interacts with the Arp2/3 complex and regulates cell migration. Nat Cell Biol 8, 1383-8 (2006).
6. Xiong, X. et al. An association between type Igamma PI4P 5-kinase and Exo70 directs E-cadherin clustering and epithelial polarization. Mol Biol Cell 23, 87-98 (2011).
7. Synek, L. et al. AtEXO70A1, a member of a family of putative exocyst subunits specifically expanded in land plants, is important for polar growth and plant development. Plant J 48, 54-72 (2006).
8. Kulich, I. et al. Arabidopsis exocyst subunits SEC8 and EXO70A1 and exocyst interactor ROH1 are involved in the localized deposition of seed coat pectin. New Phytol 188, 615-25 (2010).
9. Fendrych, M. et al. The Arabidopsis exocyst complex is involved in cytokinesis and cell plate maturation. Plant Cell 22, 3053-65 (2010).
10. Pecenkova, T. et al. The role for the exocyst complex subunits Exo70B2 and Exo70H1 in the plant-pathogen interaction. J Exp Bot 62, 2107-16 (2011).
11. Kulich, I. et al. Arabidopsis exocyst subcomplex containing subunit EXO70B1 is involved in autophagy-related transport to the vacuole. Traffic 14, 1155-65 (2013).
12. Zarsky, V., Kulich, I., Fendrych, M. & Pecenkova, T. Exocyst complexes multiple functions in plant cells secretory pathways. Curr Opin Plant Biol 16, 726-33 (2013).
13. Inoue, M., Chang, L., Hwang, J., Chiang, S. H. & Saltiel, A. R. The exocyst complex is required for targeting of Glut4 to the plasma membrane by insulin. Nature 422, 629-33 (2003).
14. Lu, H. et al. Exo70 isoform switching upon epithelial-mesenchymal transition mediates cancer cell invasion. Dev Cell 27, 560-73 (2013).
15. Liu, J., Yue, P., Artym, V. V., Mueller, S. C. & Guo, W. The role of the exocyst in matrix metalloproteinase secretion and actin dynamics during tumor cell invadopodia formation. Mol Biol Cell 20, 3763-71 (2009).
16. Drakakaki, G. et al. Clusters of bioactive compounds target dynamic endomembrane networks in vivo. Proc Natl Acad Sci USA 108, 17850-5.
17. Robert, S. et al. Endosidin1 defines a compartment involved in endocytosis of the brassinosteroid receptor BRI1 and the auxin transporters PIN2 and AUX1. Proc Natl Acad Sci USA 105, 8464-9 (2008).
18. Drakakaki, G. et al. Clusters of bioactive compounds target dynamic endomembrane networks in vivo. Proc Natl Acad Sci USA 108, 17850-5 (2011).
19. Geldner, N. et al. The Arabidopsis GNOM ARF-GEF mediates endosomal recycling, auxin transport, and auxin-dependent plant growth. Cell 112, 219-30 (2003).
20. Jaillais, Y., Fobis-Loisy, I., Miege, C., Rollin, C. & Gaude, T. AtSNX1 defines an endosome for auxin-carrier trafficking in Arabidopsis. Nature 443, 106-9 (2006).
21. Kleine-Vehn, J. et al. Differential degradation of PIN2 auxin efflux carrier by retromer-dependent vacuolar targeting. Proc Natl Acad Sci USA 105, 17812-7 (2008).
22. Robert, S. et al. ABP1 mediates auxin inhibition of clathrin-dependent endocytosis in Arabidopsis. Cell 143, 111-21 (2010).
23. Tamura, K. et al. Why green fluorescent fusion proteins have not been observed in the vacuoles of higher plants. Plant J 35, 545-55 (2003).
24. Drdova, E. J. et al. The exocyst complex contributes to PIN auxin efflux carrier recycling and polar auxin transport in Arabidopsis. Plant J (2013).
25. Chong, Y. T. et al. Characterization of the Arabidopsis thaliana exocyst complex gene families by phylogenetic, expression profiling, and subcellular localization studies. New Phytol 185, 401-19 (2010).
26. Li, S. et al. Expression and functional analyses of EXO70 genes in Arabidopsis implicate their roles in regulating cell type-specific exocytosis. Plant Physiol 154, 1819-30 (2010).
27. Hala, M. et al. An exocyst complex functions in plant cell growth in Arabidopsis and tobacco. Plant Cell 20, 1330-45 (2008).
28. Lomenick, B. et al. Target identification using drug affinity responsive target stability (DARTS). Proc Natl Acad Sci USA 106, 21984-9 (2009).
29. Lepre, C. A., Moore, J. M. & Peng, J. W. Theory and applications of NMR-based screening in pharmaceutical research. Chem Rev 104, 3641-76 (2004).
30. Duhr, S. & Braun, D. Why molecules move along a temperature gradient. Proceedings of the National Academy of Sciences of the United States of America 103, 19678-19682 (2006).
31. Seidel, S. A. et al. Microscale thermophoresis quantifies biomolecular interactions under previously challenging conditions. Methods 59, 301-15 (2013).
32. Wienken, C. J., Baaske, P., Rothbauer, U., Braun, D. & Duhr, S. Protein-binding assays in biological liquids using microscale thermophoresis. Nature Communications 1(2010).
33. Rybak, K. et al. Plant cytokinesis is orchestrated by the sequential action of the TRAPPII and exocyst tethering complexes. Dev Cell 29, 607-20 (2014).
34. Wen, T. J., Hochholdinger, F., Sauer, M., Bruce, W. & Schnable, P. S. The roothairlessl gene of maize encodes a homolog of sec3, which is involved in polar exocytosis. Plant Physiol 138, 1637-43 (2005).
35. Zarsky, V., Cvrckova, F., Potocky, M. & Hala, M. Exocytosis and cell polarity in plants—exocyst and recycling domains. New Phytol 183, 255-72 (2009).
36. Dong, G., Hutagalung, A. H., Fu, C., Novick, P. & Reinisch, K. M. The structures of exocyst subunit Exo70p and the Exo84p C-terminal domains reveal a common motif. Nat Struct Mol Biol 12, 1094-100 (2005).
37. Hamburger, Z. A., Hamburger, A. E., West, A. P., Jr. & Weis, W. I. Crystal structure of the S. cerevisiae exocyst component Exo70p. J Mol Biol 356, 9-21 (2006).
38. Moore, B. A., Robinson, H. H. & Xu, Z. The crystal structure of mouse Exo70 reveals unique features of the mammalian exocyst. J Mol Biol 371, 410-21 (2007).
39. Morris, G. M. et al. Automated docking using a Lamarckian genetic algorithm and an empirical binding free energy function. Journal of Computational Chemistry 19, 1639-1662 (1998).
40. Zhang, C., Kotchoni, S. O., Samuels, A. L. & Szymanski, D. B. SPIKE1 signals originate from and assemble specialized domains of the endoplasmic reticulum. Curr Biol 20, 2144-9 (2013).
41. Surpin, M. et al. The power of chemical genomics to study the link between endomembrane system components and the gravitropic response. Proc Natl Acad Sci USA 102, 4902-7 (2005).
42. Lewis, D. R., Miller, N. D., Splitt, B. L., Wu, G. & Spalding, E. P. Separating the roles of acropetal and basipetal auxin transport on gravitropism with mutations in two Arabidopsis multidrug resistance-like ABC transporter genes. Plant Cell 19, 1838-50 (2007).
43. Viegas, A., Manso, J., Nobrega, f. L. & Cabrita, E. Saturation-Transfer Difference (STD) NMR: A Simple and Fast Method for Ligand Screening and Characterization of Protein Binding. J Chem. Educ. 88, 990-994 (2011).
44. Drakakaki, G. et al. Isolation and proteomic analysis of the SYP61 compartment reveal its role in exocytic trafficking in Arabidopsis. Cell Res 22, 413-24 (2011).
45. Sorenson, R. & Bailey-Serres, J. Selective mRNA sequestration by OLIGOURIDYLATE-BINDING PROTEIN 1 contributes to translational control during hypoxia in Arabidopsis. Proc Natl Acad Sci USA 111, 2373-8 (2014).
46. Carter, C. et al. The vegetative vacuole proteome of Arabidopsis thaliana reveals predicted and unexpected proteins. Plant Cell 16, 3285-303 (2004).
47. Sohn, E. J. et al. The shoot meristem identity gene TFL1 is involved in flower development and trafficking to the protein storage vacuole. Proc Natl Acad Sci USA 104, 18801-6 (2007).
48. Lomenick, B., Jung, G., Wohlschlegel, J. A. & Huang, J. Target identification using drug affinity responsive target stability (DARTS). Curr Protoc Chem Biol 3, 163-180 (2012).
49. Emsley, P. & Cowtan, K. Coot: model-building tools for molecular graphics. Acta Crystallogr D Biol Crystallogr 60, 2126-32 (2004).
50. Adams, P. D. et al. PHENIX: building new software for automated crystallographic structure determination. Acta Crystallogr D Biol Crystallogr 58, 1948-54 (2002).
51. Pedretti, A., Villa, L. & Vistoli, G. VEGA—An open platform to develop chemo-bio-informatics applications, using plug-in architecture and script programming. Journal of Computer-Aided Molecular Design 18, 167-173 (2004).
52. Gasteiger, J. & Marsili, M. Iterative Partial Equalization of Orbital Electronegativity—a Rapid Access to Atomic Charges. Tetrahedron 36, 3219-3228 (1980).
53. Morris, G. M. et al. AutoDock4 and AutoDockTools4: Automated Docking with Selective Receptor Flexibility. Journal of Computational Chemistry 30, 2785-2791 (2009).

Although the present invention has been described in connection with the preferred embodiments, it is to be understood that modifications and variations may be utilized without departing from the principles and scope of the invention, as those skilled in the art will readily understand. Accordingly, such modifications may be practiced within the scope of the invention and the following claims.

1. A method of altering exocytosis and/or endocytic recycling in a plant, fungal or animal cell, comprising
exposing the cell to a compound that modifies exocytosis and/or endocytic recycling by binding to an exocyst complex or to an exocyst complex subunit of the cell.

2. The method of claim 1, wherein the cell is in a subject in need of treatment for diabetes or cancer, and
an effective amount of the compound is administered to the subject to treat the diabetes or cancer.

3. A method of screening for a compound for altering exocytosis and/or endocytic recycling, comprising
in a cell-free system, detecting or measuring binding of a test compound to an exocyst complex or to a subunit of an exocyst complex.

4. The method of claim 1, wherein the subunit is an EXO70 protein isoform.

5. The method of claim 4, wherein the EXO70 protein isoform is EXO70A1.

6. The method of claim 1, wherein the compound promotes or inhibits exocyst complex activity.

7. The method of claim 1 wherein the compound binds to the C-terminal portion of EXO70A1.

8. The method of claim 7, wherein the compound binds to a cavity in the C-terminal portion of EXO70A1.

9. The method of claim 1, wherein the compound is Endosidin2 (ES2) or an analog thereof.

10. The method of claim 9, wherein the analog is an N′-benzylidenebenzohydrazide analog of compound ES2, wherein the N′-benzylidenebenzohydrazide analog comprises:
(i) a substituted or non-substituted iodine-containing phenyl group of ES2; or
(ii) a substituted or non-substituted fluorine-containing benzoic ring of ES2; or
(iii) both (i) and (ii).

11. The method of claim 10, wherein the benzoic ring lacks the fluorine present in ES2.

12. The method of claim 10, wherein the N′-benzylidenebenzohydrazide analog binds to EXO70A1.

13. The method of claim 10, wherein the N′-benzylidenebenzohydrazide analog promotes or inhibits exocyst complex activity.

14. The method of claim 13, wherein the N′-benzylidenebenzohydrazide analog alters exocytosis and/or endocytic recycling in a plant, fungal or animal cell.

15. The method of claim 14, wherein the N′-benzylidenebenzohydrazide analog inhibits exocytosis and/or endocytic recycling in the plant, fungal or animal cell.

16. An N′-benzylidenebenzohydrazide analog of compound ES2, wherein the analog comprises:
(i) a substituted or non-substituted iodine-containing phenyl group of ES2; or
(ii) a substituted or non-substituted fluorine-containing benzoic ring of ES2; or
(iii) both (i) and (ii).

17. The analog of claim 16, wherein the benzoic ring lacks the fluorine present in ES2.

18. The analog of claim 16, wherein the analog binds to EXO70A1.

19. The analog of claim 16, wherein the analog promotes or inhibits exocyst complex activity.

20. The analog of claim 19, wherein the analog alters exocytosis and/or endocytic recycling in a plant, fungal or animal cell.

21. The analog of claim 20, wherein the analog inhibits exocytosis and/or endocytic recycling in the plant, fungal or animal cell.
